(19) United States
(12) Patent Application Publication
(10) Pub. No.: US 2016/0242894 A1
(43) Pub. Date: Aug. 25, 2016

(54) URETHRAL PATENCY IMPLANT

(71) Applicant: Scaffold Medical AG, Zurich (CH)

(72) Inventor: H. Richard Davis, Coral Springs, FL (US)

(21) Appl. No.: 15/047,305

(22) Filed: Feb. 18, 2016

Related U.S. Application Data
(60) Provisional application No. 62/118,942, filed on Feb. 20, 2015.

(51) Int. Cl. A61F 2/04; A61F 2/915; A61F 2/844

(52) U.S. Cl. CPC A61F 2/04; A61F 2/844; A61F 2/915; A61F 2002/047; A61F 2002/9155; A61F 2210/0076; A61F 2230/0069; A61F 2250/0059; A61F 2250/0071

(57) ABSTRACT

A urethral scaffold is provided for expanding a portion of the urethra that extends through the prostate. The scaffold is expandable and has a plurality of undulating circumferential members disposed between a proximal end and a distal end of the scaffold. A central section of the scaffold has a width that exceeds the width at the distal end of the scaffold. The scaffold is made of a material that reacts with urine or with tissue surrounding the urethra to reduce the volume of the scaffold such that the scaffold can self-explant without an interventional procedure.

### BACKGROUND OF THE INVENTION

1. Field of the Invention

This application is directed to therapies for prostate cancer and/or benign hyperplasia of the prostate using an implant configured to enhance patency in a urethra.

2. Description of the Related Art

Obstruction of the urethra is a common medical problem for men. The obstruction is commonly caused by enlargement of the prostate. The prostate is a gland that surrounds the urethra and that has a role in the reproductive system. Over time, the prostate can become enlarged due to cancer, accumulation of benign cells, trauma, infection and for other reasons. As cancer or benign cells proliferate, the volume of the prostate enlarges causing the flexible tissue of the urethra, which passes through the prostate, to collapse, restricting flow in the urinary tract.

Treatment options for cancer or benign prostate hyperplasia (BPH) are limited. Cancer can be treated by surgery, radiation, chemotherapy and other modes. If the condition is BHP, the growth of the prostate is often slow and becomes noticeable later in life. As a result, many clinicians counsel “watchful waiting” where the patient tolerates the symptoms, while being subject to periodic blood tests and biopsy procedures to monitor changes in the condition. Patients whose symptoms are more disruptive may be treated with more aggressive therapies, such as surgery or transurethral procedures such as transurethral microwave therapy (TUMT) or transurethral needle ablation (TUNA).

Whatever the cause, a common technique to restoring flow in the urethra is to insert a balloon catheter through the urethra from the external opening thereof into the bladder. The balloon, which is located at the distal end of the catheter, is inflated inside the bladder to retain the catheter in the body. The catheter body size must accommodate a first fluid flow channel for balloon inflation and a second fluid flow channel for the outflow of urine. This catheter is often left in place from days to weeks and is a significant inconvenience and, for many patients, a source of bleeding and discomfort. If not properly maintained, such catheters can be a source of infection. Also, retaining a catheter along the length of the urethra can lead to incontinence.

Efforts have been made to develop implants for improving the patency of the urethra. Such efforts have generally failed to produce any commercial products for a number of reasons. A primary problem in past efforts has been progressive growth of deposits from the urine, or encrustation, on the structure. These deposits eventually were a source of obstruction equal or greater to the obstruction the devices sought to treat. Also, some implants were unable to retain their position in the body, resulting in migration. In some cases, the migration caused severe complications.

### SUMMARY OF THE INVENTION

There is a need for new implants for the urinary tract that will provide patency and thus more normal unobstructed flow of urine. In some embodiments, the implants are temporary. Preferably the implants are configured to be self-explanting or self-removing from the patient. The processes for self-explantation can include one or more of erosion, dissolving, reacting, and/or absorbing into the patient.

The scaffold is configured to hold open a lumen, as discussed below. The scaffold is configured to be implanted in the urethra in the region of the prostate. The scaffold can be temporary, remaining in the urethra for a set period, such as thirty days.

In a specific embodiment, the scaffold is bioerodable, which in this context means that the volume of the scaffold is slowly reduced as small portions are absorbed in, dissolved in or otherwise carried away by the urine. However, the scaffold is configured to retain its rigidity over time even as its volume is reduced. The analogy is to a piece of hard candy that remains hard as its volume reduces.

In one embodiment the scaffold is made of or contains a significant amount of magnesium such that when exposed to urine it emits hydrogen gas as it erodes. The gas is carried by the urine out of the patient. This material is absorbed into urine at a rate that allows it to retain sufficient structural integrity over a period of up to ninety days, of seventy-five days or less, of up to sixty days, of at least about forty-five days, of thirty days or less, or of up to a week.

The scaffold can be coated to modulate the erosion process. The coating can be polymer that initially completely encapsulates the scaffold to prevent it from contacting the urine. The coating can be configured to be biodegradable, such that over a period, e.g., over thirty days, it gradually exposes all or portions of the scaffold to permit the erosion process to commence. The coating need not retain rigidity and can more rapidly degrade than the scaffold.

The coating can be loaded with a drug to treat the prostate to reduce or eliminate re-growth or re-encroachment of the prostate into the passage of the urethra.

In one variation, a scaffold is provided to expand a portion of a urethra that extends through a prostate. The scaffold has an elongate body that has a proximal end, a distal end, an outer surface, and an inner surface. The elongate body has a plurality of undulating circumferential members disposed between the proximal end and the distal end. The undulating circumferential members are spaced apart along a longitudinal axis of the scaffold. The undulating circumferential members are connected to at least one adjacent undulating circumferential member by at least one axial connector. The elongate body has a collapsed state and an expanded state. The collapsed state is configured to enable the elongate body to be delivered into the urethra and to enable the elongate body to be navigated to a position within the portion of the urethra that extends through the prostate. The expanded state is configured such that the undulating circumferential members provide the elongate body with sufficient radial strength to maintain open the portion of the urethra that extends through the prostate. The elongate body having a width defined in a plane transverse to the longitudinal axis at a distal-end, the elongate body has a width defined in a plane transverse to the longitudinal axis at a central section located between the distal end and the proximal end. The width at the central section exceeds the width at the distal end. The elongate body has a material that reacts with urine or with the tissue surrounding the urethra to reduce the volume of the elongate body such that the scaffold can be removed from the urethra after a prescribed period without requiring an interventional procedure. In some variants, at least a portion of the elongate body is configured to be eroded by urine and/or absorbed into tissue surrounding the urethra.

In another embodiment, a scaffold is provided for expanding the urethra through the prostate. The scaffold has an elongate body that has a proximal end, a distal end, an outer surface, and an inner surface. The elongate body is disposed along a longitudinal axis of the scaffold between the proximal end and the distal end. The elongate body has a collapsed state and an expanded state. The elongate body has sufficient radial strength to maintain a lumen thereof open when the elongate body is disposed in the prostate region of the urethra. The outer surface of the elongate body has a width defined in a plane transverse to the longitudinal axis at a distal-end, the outer surface of the elongate body having a width defined in a plane transverse to the longitudinal axis at a central section located between the distal end and the proximal end. The width at the central section is greater than the width at the distal end. The elongate body comprises a removal configuration in which the scaffold will self-explant after a prescribed period.

In another embodiment, a scaffold for is provided for expanding a urethra through the prostate. The scaffold includes an elongate body that has a proximal end, a distal end, an outer surface to be expanded into contact with the urethra, and an inner surface configured to surround a lumen for urine flow. The elongate body has an expanded state configured with sufficient radial strength to maintain the lumen open when disposed in the prostate region of the urethra that is occluded by an enlarged prostate. The inner surface is configured to be eroded by urine.

The scaffold can be delivered using a delivery system that includes an anchor and guide catheter and a scaffold deployment catheter. The anchor and guide catheter has sufficient stiffness to push through the urethra but has a soft distal end such that interactions with the inside of the bladder are blunt. The soft distal end has an anchor portion that can include a soft balloon. For example, a latex balloon can be used. In general the anchor member preferably is able to expand to a high volume (diameter) at relatively low pressure.

The anchor portion is mounted on a small diameter tubular member that is small enough to permit a balloon catheter, e.g., having about a 0.035″ shaft, to track thereover. The tubular member has a single lumen in one embodiment for inflation of the balloon. The tubular member has a second lumen in one embodiment to permit drainage of the bladder during a treatment.

The scaffold deployment catheter is moveable, slideable or telescoped over the anchor system in various embodiments. The scaffold can be crimped down to a 5 mm diameter on a balloon of the scaffold deployment catheter in one embodiment.

In another embodiment, a system is provided for treating a urethra. The system includes a scaffold delivery assembly and a temporary urethral scaffold. The scaffold delivery assembly comprises a scaffold delivery catheter that includes an elongate body having a proximal end, a distal end, and a central lumen disposed between the proximal and distal ends. The scaffold delivery catheter includes a deployment balloon disposed on a side surface of the elongate body. The deployment balloon is in fluid communication with an inflation lumen disposed in the elongate body between the proximal end and the deployment balloon. The temporary urethral scaffold has a material configured to react with urine and/or tissue disposed around the urethra to cause the scaffold to erode over time and to self-explant after a prescribed period.

In some aspects, the system includes an anchor balloon disposed at a distal end of the system. In certain embodiments, the anchor balloon is disposed at a distal end of an anchor balloon catheter that has an elongate body coupled with and extending proximally from the anchor balloon catheter. The elongate body is slideably disposed in a lumen of the scaffold delivery catheter and configured to convey inflation media to the anchor balloon. In certain aspects, the anchor balloon catheter has a lumen extending from a distal end to a proximal end of the anchor balloon catheter and configured to convey urine out of the patient during a procedure or during recovery.

The invention involves methods of implanting as well. In a clinical setting, the medical personnel track the anchor/guide catheter through the urethra into the bladder, to the back of the bladder. The anchor member is expanded, e.g., the balloon is inflated. The anchor member is pulled back into contact with the wall around the urethra. Then the scaffold deployment catheter is advanced over the elongate member of the anchor/guide catheter until the distal end of the scaffold deployment catheter abuts the proximal face of the balloon (or other anchor member). The balloon on the scaffold deployment catheter is inflated to expand the scaffold. In certain embodiments, the position of the scaffold is confirmed before inflating the balloon on the scaffold deployment catheter by viewing the position of one or more marker bands disposed at a known position relative to the scaffold. In some aspects, the one or more marker bands include a radiopaque material. In some embodiments, expanding the scaffold includes expanding a plurality of undulating circumferential members spaced apart along the length of the scaffold.

The balloon on the scaffold deployment catheter and the anchor/guide catheter are withdrawn and the system removed from the urethra leaving the scaffold in place.

In one example method, a delivery catheter is advanced into a urethra. The delivery catheter has a scaffold coupled therewith. The scaffold has a proximal end, a distal end, and an elongate body disposed therebetween. The elongate body includes a central portion of the scaffold. The scaffold is positioned such that the distal end is adjacent to an end of the prostate closest to the bladder and the proximal end is adjacent to an end of the prostate farthest away from the bladder. The position of the scaffold within the prostate is confirmed. The scaffold is expanded away from a longitudinal axis of the catheter into apposition with the tissue surrounding the urethra. The central portion of the elongate body is enlarged by a greater amount than at least one of the proximal end and the distal end of the scaffold. The scaffold reduces the constriction of the urethra within the prostate.

Further methods involve activating the bioerosion of the scaffold. In one method, the activation is programmed into a coating as discussed above. The coating degrades in urine eventually exposing the scaffold material (e.g., magnesium) at which point erosion can commence.

In another embodiment, a subsequent action causes erosion. For example, another catheter can be inserted into the volume of the scaffold and brought into contact with the scaffold to nick the coating exposing the underlying layer to commence erosion. In certain embodiments, an erosion accelerant is delivered through the distal portion of the elongate catheter body into the urethra adjacent to the inner surface of the scaffold. In some aspects, the erosion accelerant is delivered by inflating a balloon with an inflation medium disposed on the distal portion of the elongate catheter body and maintaining a pressure in the balloon while the inflation medium flows out of a surface of the balloon and onto the inner surface of the scaffold.

In another example method, a urethral scaffold is explanted by advancing into a urethra a distal portion of an elongate catheter body having a snare extending within a lumen of the distal portion of the catheter. The distal portion of the catheter is brought adjacent to a proximal end of the scaffold. The scaffold has an outer surface disposed against the urethra and a snare feature disposed inward of the outer surface. An arcuate portion of the snare is advanced from the catheter body and engages the snare feature of the scaffold. The snare feature of the scaffold is moved relative to the distal portion of the catheter body to compress the scaffold into the lumen of the catheter body. In certain embodiments, the scaffold has a tapered proximal portion that is configured to be at least partially received in the lumen of the catheter body before the scaffold begins to compress.

### BRIEF DESCRIPTION OF THE DRAWINGS

These and other features, aspects and advantages are described below with reference to the drawings, which are intended to illustrate but not to limit the inventions. In the drawings, like reference characters denote corresponding features consistently throughout similar embodiments. The following is a brief description of each of the drawings.

FIG. 1A is a schematic of a normal human bladder, prostate and portion of a urethra.

FIG. 1B is a schematic of a human bladder and urethra, with an enlarged prostate disposed around the urethra, the enlarged prostate causing constriction and obstruction of the segment of the urethra extending through the prostate.

FIG. 2 shows a scaffold according to the present application disposed in the segment of the urethra extending through the prostate, the scaffold enlarging the urethra compared to a diseased state to improve flow and reduce discomfort.

FIG. 3 is a perspective view of the scaffold illustrated in the body in FIG. 2, showing high level features.

FIG. 3A shows a schematic cross-section illustrating a delivery profile for one embodiment of the scaffold of FIG. 3.

FIG. 3B shows a schematic cross-section illustrating an expanded profile for one embodiment of the scaffold of FIG. 3.

FIG. 3C shows a wall pattern for one embodiment of a scaffold, with the pattern shown in a flat configuration.

FIG. 3D shows a wall pattern for another embodiment of a scaffold, with the pattern shown in a flat configuration.

FIG. 3E shows three embodiments of strut configurations for various embodiments of the scaffold of FIG. 3.

FIG. 3F is a perspective view of a variation of the scaffold illustrated in the body in FIG. 2, illustrating a layered construction.

FIG. 4 shows a system for delivering a scaffold configured for treating urethral obstruction.

FIGS. 4A-4C shows other systems for delivering a scaffold configured for treating urethral obstruction, the system not requiring a separately slideable anchor balloon catheter;

FIG. 5 illustrates an anchor catheter of the delivery system of FIG. 4.

FIG. 6 illustrates the anchor catheter of FIG. 5 with an anchor balloon thereof expanded to enable the delivery system FIG. 4 to be retained in a urinary tract of a patient.

FIG. 7 illustrates a deployment catheter of the delivery system of FIG. 4, the deployment catheter being adapted to advance over the anchor catheter of FIG. 5 to deploy a scaffold in a urethra.

FIG. 8 illustrates the deployment catheter of FIG. 7 with an expandable member in an expanded configuration, the expanded configuration causing the scaffold to be expanded.

FIG. 8A shows an embodiment of a deployment catheter configured to expand a scaffold to a configuration with varying width or diameter.

FIG. 9A illustrates a balloon catheter that includes an expandable member configured to allow an inflation media to seep therefrom as part of a simultaneous treatment or a subsequent step for removal.

FIG. 9B illustrates a balloon catheter configured to expedite removal of a scaffold.

FIG. 10 illustrates a retrieval device configured to be placed in a scaffold to expedite removal thereof from the urethra.

FIG. 11 illustrates placement and expansion of the anchor balloon of the anchor catheter of FIG. 5 in a bladder.

FIG. 12 illustrates advancement of a delivery catheter through the urethra over the elongate body of the anchor catheter such that an expansion member having a scaffold mounted thereon is disposed proximal of the downstream sphincter muscle just outside a length of the urethra surrounded by the prostate.

FIG. 13 illustrates advancement of the delivery catheter over the elongate body of the anchor catheter to a location between the sphincter muscles such that the expansion member and the scaffold are disposed proximal within a length of the urethra surrounded by the prostate.

FIG. 14 illustrates expansion of the expandable member of the delivery catheter in the position shown in FIG. 13, the expandable member expanding a scaffold into apposition with the inside surface of the urethra.

FIG. 15 shows a completed procedure with the delivery system removed and the scaffold is left in place.

### DETAILED DESCRIPTION OF THE PREFERRED EMBODIMENT

While the present description sets forth specific details of various embodiments, it will be appreciated that the description is illustrative only and should not be construed in any way as limiting. Furthermore, various applications of such embodiments and modifications thereto, which may occur to those who are skilled in the art, are also encompassed by the general concepts described herein. Each and every feature described herein, and each and every combination of two or more of such features, is included within the scope of the present invention provided that the features included in such a combination are not mutually inconsistent.

### I. Urethral Lumen Blockage and Flow Restoration

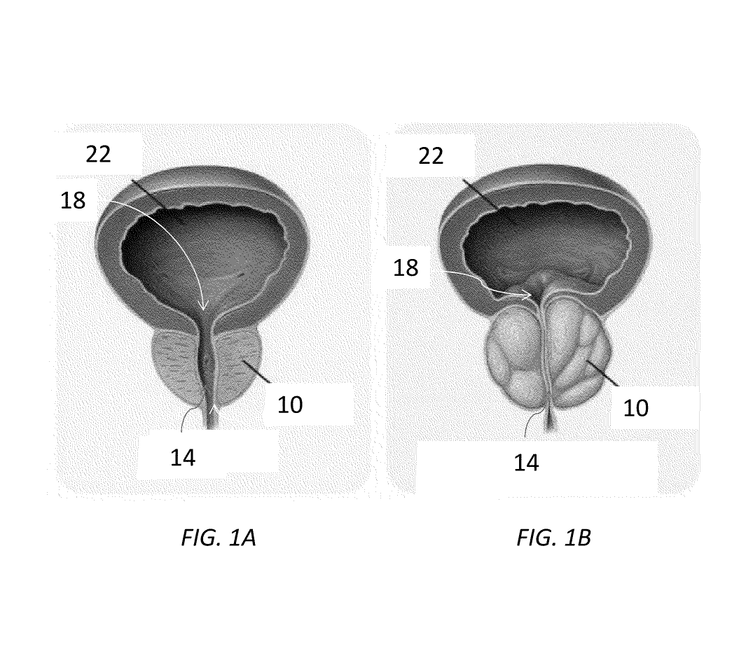

FIGS. 1A and 1B depict a human prostate 10 and a urethra 14 that extends through the prostate 10. The urethra 14 has an opening 18 at the neck of the bladder 22 and an external opening (not shown) through which urine leaves the body. A length of the urethra 14 between the opening 18 and the external opening is unobstructed in the normal urinary tract (FIG. 1A) but is massively obstructed in a urinary tract with advanced prostate cancer or advanced BHP (FIG. 1B).

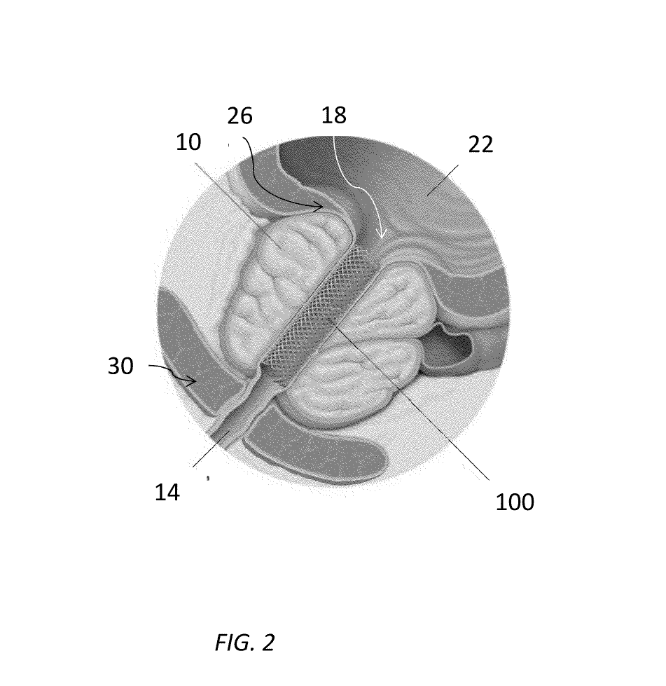

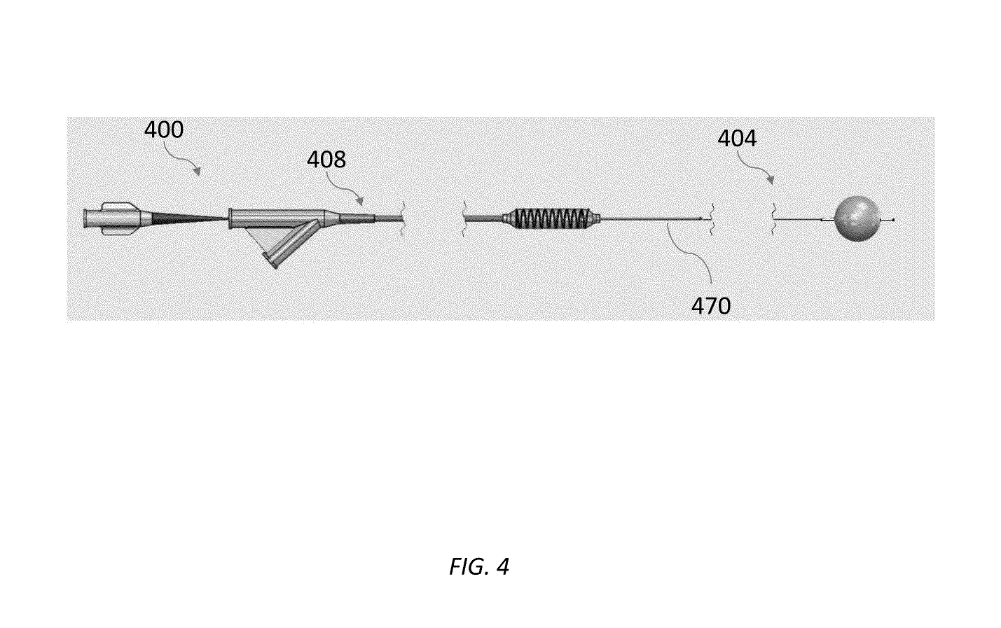

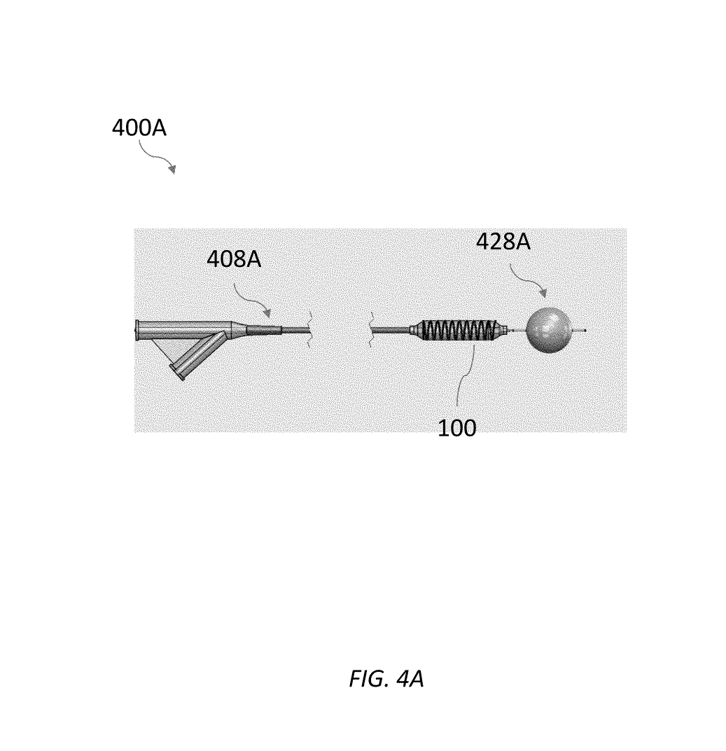

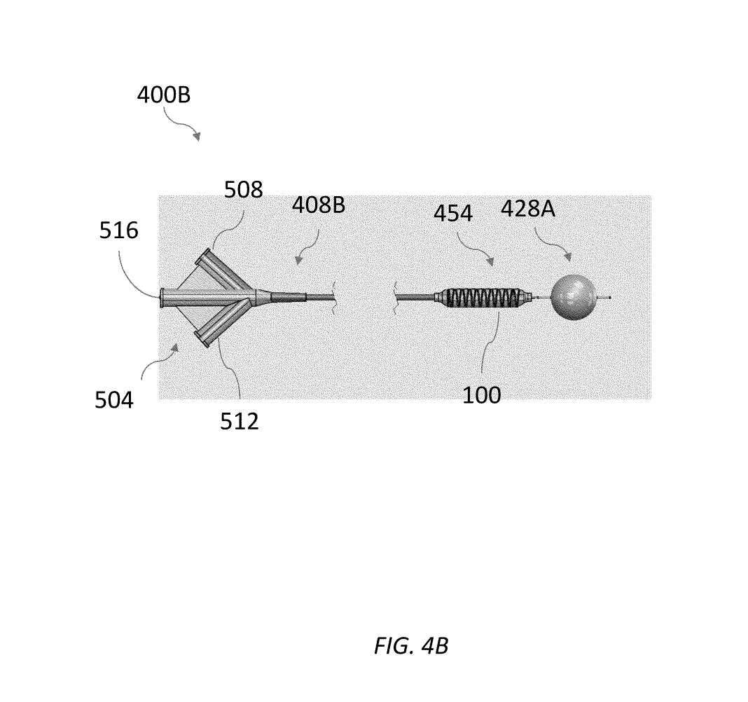

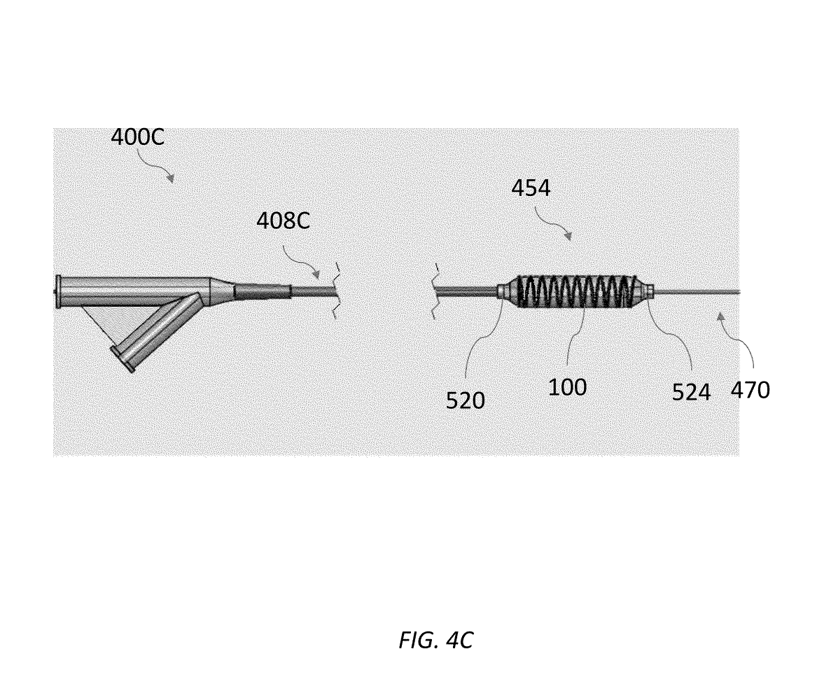

FIG. 2 shows a scaffold 100 placed in the urethra 14 within the prostate 10. The scaffold 100 is placed in the urethra 14 downstream of the opening 18 from the bladder 22. The scaffold 100 is placed such that is not obstructing the function of an upstream sphincter muscle 26. The scaffold 100 also is placed such that it is not obstructing a downstream sphincter muscle 30. Careful placement of the scaffold 100 can be provided by various delivery systems. For example, a two catheter is discussed in connection with FIGS. 4-8 and 11-14.

When the scaffold 100 is placed as shown in FIG. 2, the obstruction of the urethra 14 illustrated in FIG. 1B is reduced or eliminated to provide an open condition as illustrated in FIG. 1A. The scaffold 100 also provides minimal to no interference with the valve function of the muscles 26, 30 providing natural or near natural regulation of flow. As discussed in various embodiments below the scaffold 100 can be temporary in various applications. For example, certain patients are expected to undergo surgery. The scaffold 100 can be used instead of a catheter to facilitate draining the bladder while the patient's procedure is scheduled. This provides the advantages of being completely internal and not interfering with the muscles, 26, 30. For other patients, infection or other transitory conditions cause the impingement. No surgery is needed, just temporary relief of the urethral obstruction until swelling subsides. Such patients could be treated with a catheter, but the scaffold 100 provides the advantages of being internal and not interfering with the muscles 26, 30. The scaffold 100 can also be a vehicle to directly administer an agent for reducing the swelling, as discussed below. Various embodiments of the scaffold can be self-removing or self-explanting. This is another advantage over a conventional bladder catheter. They can be expelled from the penis intact or in small sections after a prescribed time or can be triggered to be removed or even grasped if such a course of action is deemed appropriate.

### II. Scaffolds for Urethral Flow Restoration

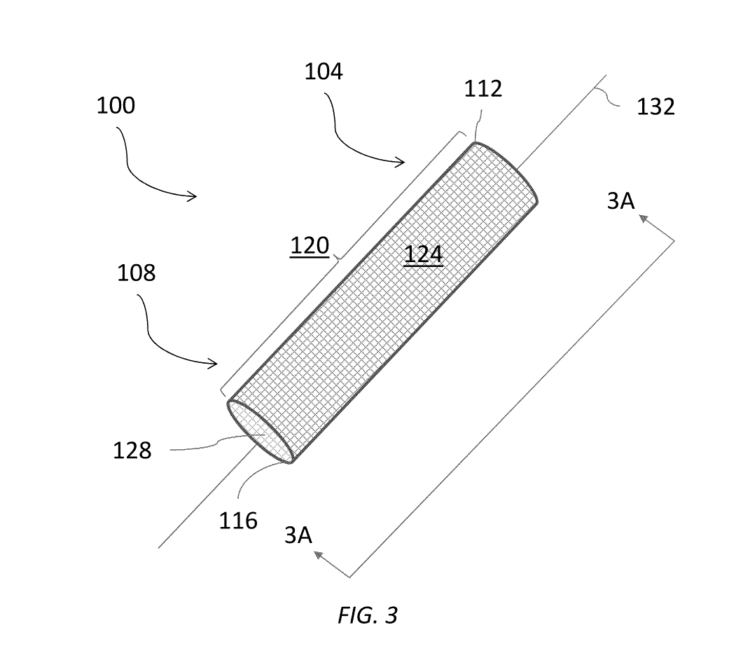

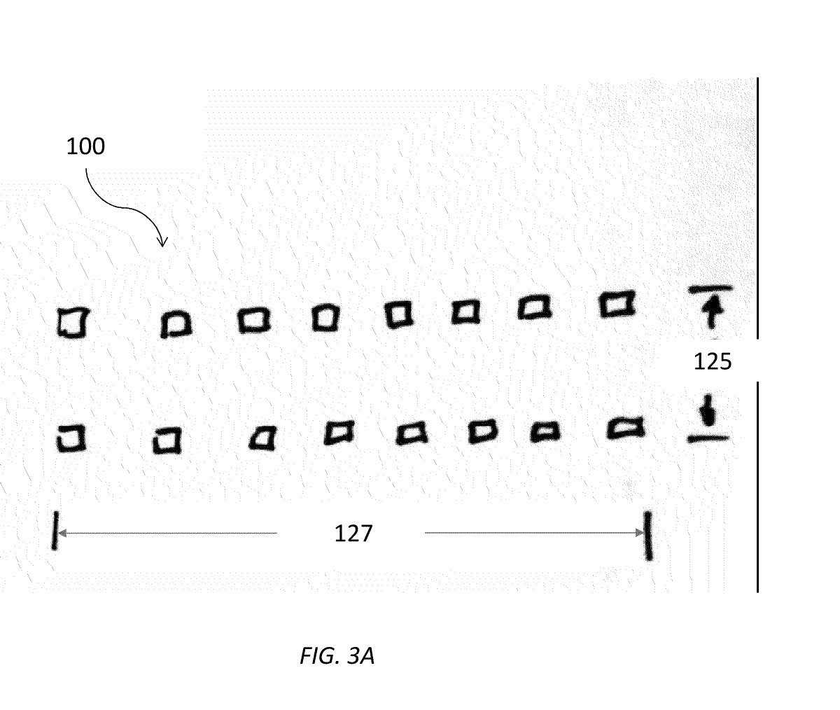

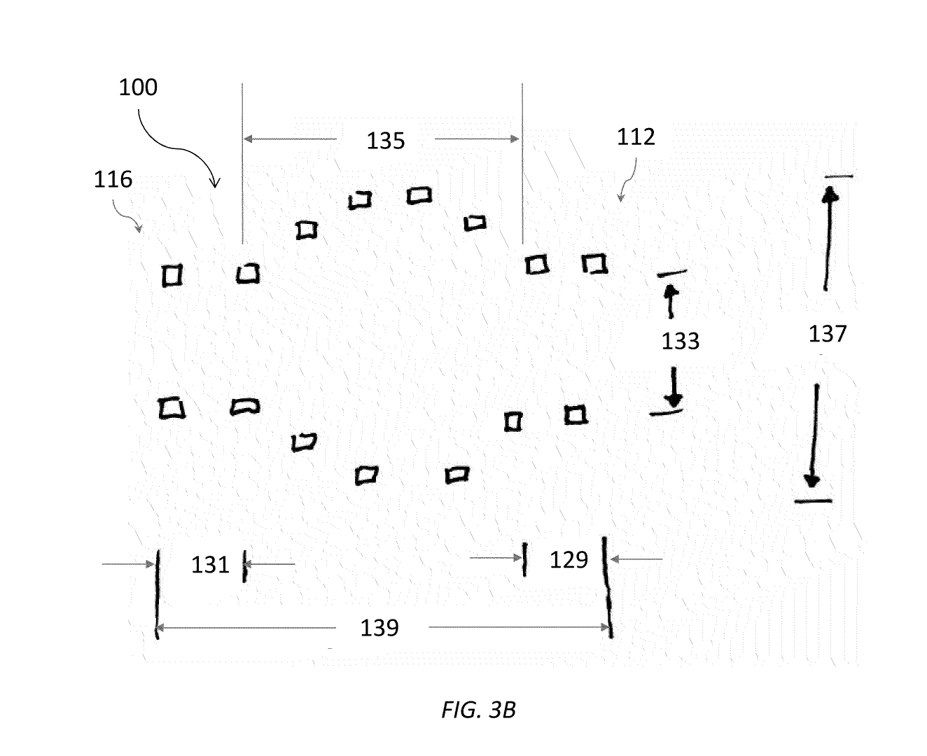

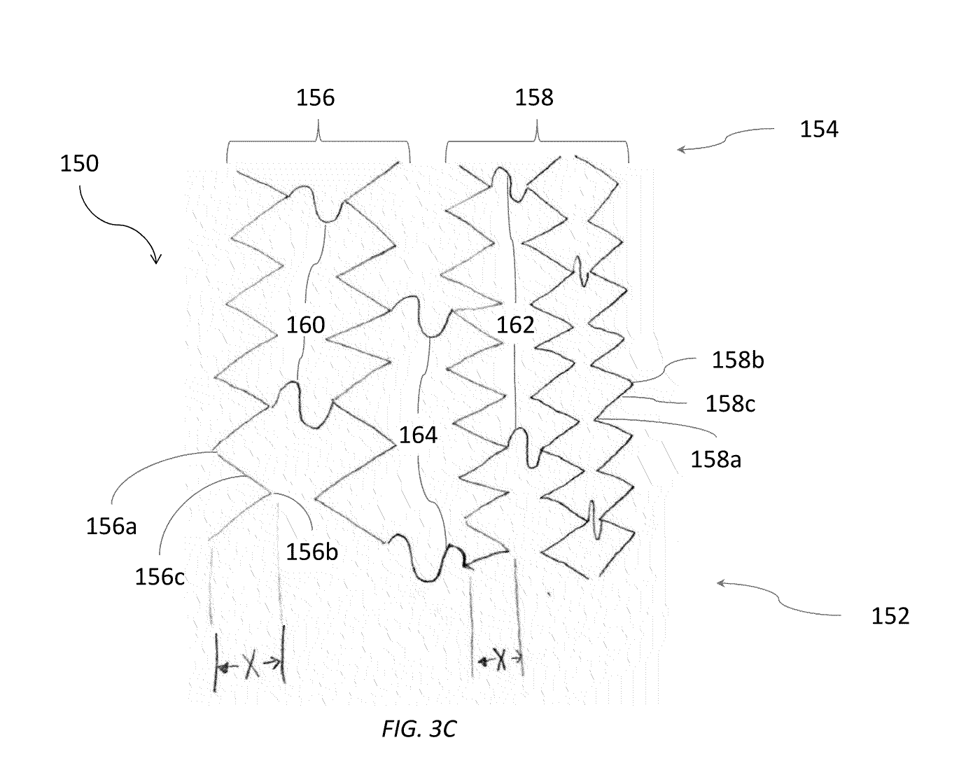

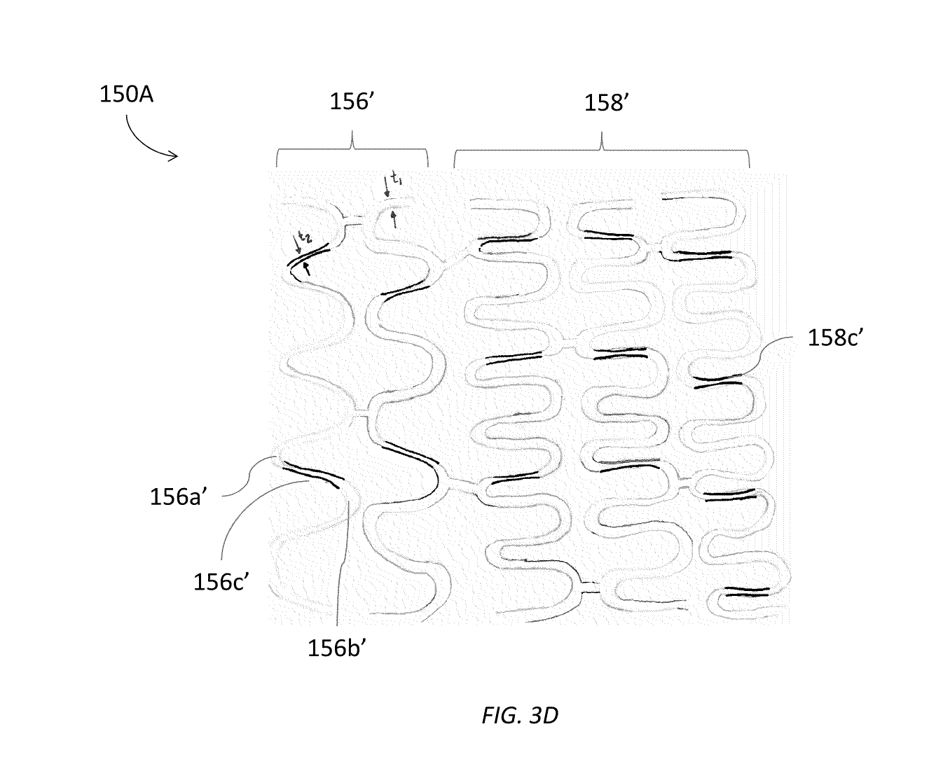

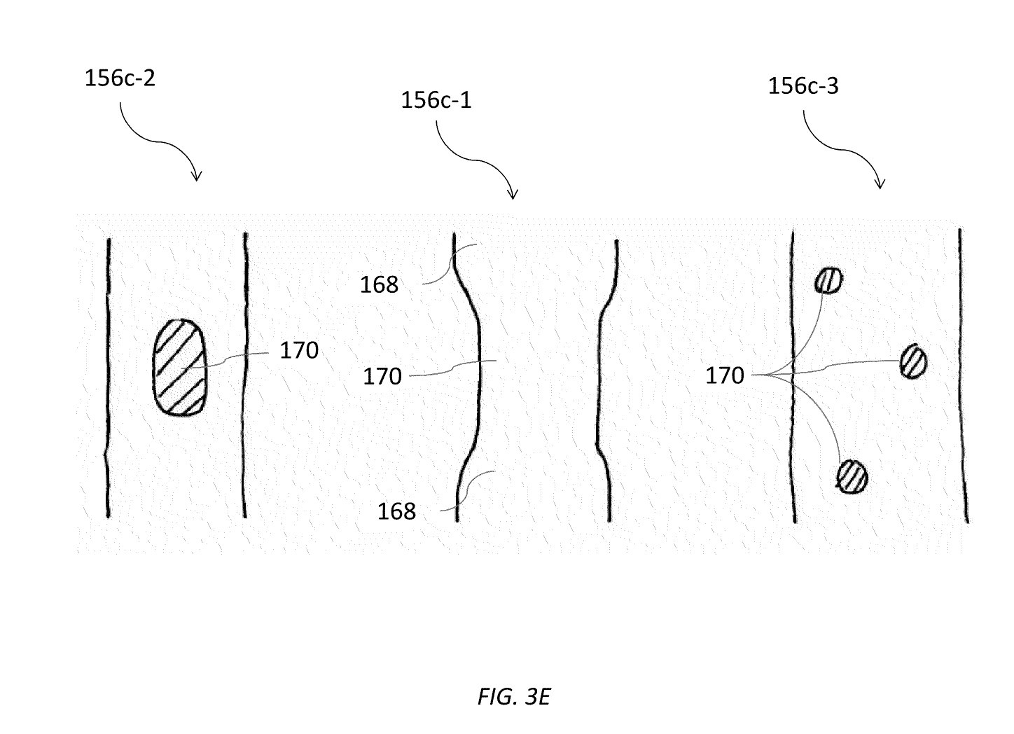

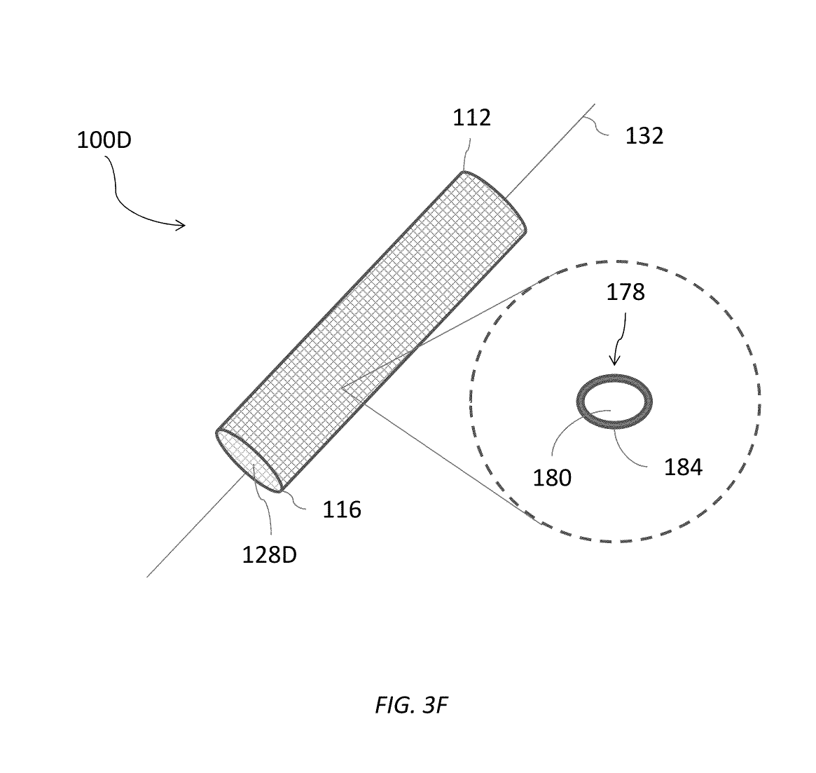

FIG. 3 shows details of various embodiments of the scaffold 100. The scaffold 100 includes a distal portion 104 and a proximal portion 108. The distal portion 104 can include a distal end 112 of the scaffold 100. The proximal portion 108 can include a proximal end 116 of the scaffold 100. The scaffold 100 includes an elongate body 120 that extends between the distal portion 104 and the proximal portion 108. The elongate body 120 can extend from the distal end 112 to the proximal end 116.

The elongate body 120 has an outer surface 124 and an inner surface 128. The outer surface 124 is configured have an outer perimeter that is large enough to hold body tissues away from the urethra 14 such that the inner surface 128 can allow for flow through elongate body 120. The scaffold 100 preferably has sufficient radial strength to displace the encroaching prostate 10 illustrated in FIG. 1B away from the urethra 14. The inner surface 128 of the elongate body 120 is configured in some embodiments to have an inner perimeter that is about the size of a human urethra when not narrowed by enlargement of the prostate 10 as discussed above. For example the inner perimeter of the scaffold 100 can be circular with a diameter of 10 mm or less, e.g., about 9 mm, in some cases about 8 mm or less, or between 7 mm and 8 mm, about 7 mm, in some cases about 6 mm.

The elongate body 120 is disposed along a longitudinal axis 132 of the scaffold 100 between the proximal end 116 and the distal end 112. FIG. 3 shows that in some embodiments, the elongate body 120 can have a generally constant profile. In other embodiments, the profile of the elongate body 120 can vary at least in the expanded state along the length of the body 120. In some variations discussed below, the scaffold 100 has different widths at different locations between the proximal end 116 and the distal end 112 when expanded. For example the expanded size can vary between 2 mm and 9 mm along the length of the elongate body 120 in one embodiment. The larger widths can be disposed in a central portion of the scaffold 100. The scaffold 100 is preferably configured to expand to have and to retain an enlarged mid-portion to aid in retention of the scaffold 100, yet is configured to avoid interfering with the valve function of the muscles 26, 30. The radial strength of the elongate body 120 is preferably at least about 7 psi to about 10 psi in some embodiments. In other embodiments, the radial strength of the elongate body 120 is about 5 psi to about 8 psi. In other embodiments, the radial strength of the elongate body 120 is about 7 psi.

As discussed further below, there are a number of ways to implant the scaffold 100 in a patient. In one embodiment, the scaffold 100 is configured such that it can be advanced transurethrally in a collapsed state. The elongate body 120 can have a collapsed state and an expanded state to facilitate this delivery approach. The collapsed state is discussed below in connection with the delivery system 400. The collapsed state is one in which the scaffold 100 has a smaller transverse dimension such that it can be advanced with reduced or minimal abrasion or irritation of the tissue of the urethra 14. FIG. 3A illustrates a collapsed state in which the elongate body 120 has a circular outer periphery with a substantially constant diameter along the length of the elongate body 120 between the distal end 112 and the proximal end 116. The width 125 and length 127 of the elongate body 120 can vary depending on the condition being treated. In one embodiment, the width 125 is less than about 5 mm, e.g., about 4 mm or less, in some cases about 3 mm and in some cases about 2 mm. The length 127 of the elongate body can be about 60 mm or less, in some cases between about 25 mm and about 55 mm, or between about 30 mm and about 50 mm, in other instances between about 35 mm and 45 mm. Specific embodiments provide that the length 127 of the elongate body 120 of the scaffold is about 35 mm, about 45 mm, or about 55 mm.

FIG. 3B illustrates an expanded state of one embodiment of the scaffold 100 in which the elongate body 120 has a varying profile along the length of the body 120. In the varying profile, the transverse cross-section of the elongate body 120 is larger at locations spaced from the distal end 112. The varying profile can be one in which the transverse cross-section is larger at locations spaced from the proximal end 116. The elongate body can be enlarged in transverse cross-section at locations spaced from both the distal end 112 and the proximal end 116. The outer surface 124 of elongate body 120 can surround a space having a width dimension defined in a plane transverse to the longitudinal axis 132 at a distal end 112. The outer surface 124 can surround a space having a width dimension defined in a plane transverse to the longitudinal axis 132 at a central section located between the distal end 112 and the proximal end 116. The width dimension at the central section can exceed the width dimension at the proximal end 116. The width dimension at the central section exceeds the width dimension at the distal end 112. The width dimension at the central section exceeds the width dimension at the proximal end 116 and at the distal end 112.

In one embodiment, the scaffold 100 has an expanded state in which a distal zone 129 has a first profile 133A. The distal zone 129 extends proximally from the distal end 112. A proximal zone 131 has a second profile 133B. The proximal zone 133 extends distally from the proximal end 116. A central zone 135 can be provided in one embodiment. The central zone 135 can extend between the distal zone 129 and the proximal zone 131. In some embodiments, the central zone 135 extends from the distal zone 129 to the proximal zone 131. In some embodiments, other zones are provided between the central zone 135 and at least one of the distal zone 129 and the proximal zone 133. The central zone 135 can have a third profile 137. The first profile 133A can be the same as the second profile 133B. In other embodiments the first profile 133A is configured to lessen the risk of incontinence by providing a smaller diameter at the distal end 112 that is provided at the proximal end 116. In other embodiments the first profile 133A is configured to lessen the risk of incontinence by providing a taper from a distal portion of the central zone 135 to the distal end 112. In other the risk of incontinence is lessened by configuring the first profile 133A with a larger slope than the second profile 133B such that the effect on the sphincter muscle 30 is less than that on the sphincter muscle 26.

In one embodiment the first profile 133A has a width as measured in a cross-section transverse to the longitudinal axis 132 of about 5 mm, e.g., about 6 mm or more, in some cases about 7 mm, about 8 mm, about 9 mm, or about 10 mm or more. In many embodiments the elongate body 120 is circular in transverse cross-section and the width dimensions are diameters. The second profile 133B can match or be different from, e.g., larger than, the first profile 133A. The third profile 137 can be selected to provide one more function such as to provide sufficient opening of the urethra 14 at the central zone 135. In some embodiments, the central zone 135 is enlarged more than necessary to provide sufficient flow. For example the central zone 135 can be enlarged sufficiently to remodel the prostate over a prescribed treatment period. This can be accomplished by a method that includes over-expanding the prostate 10. The central zone 135 can be enlarged sufficiently to provide a retention effect for the scaffold 100 in the prostate 10. In some cases, the large third profile 137 provides both the benefit of sufficient retention of the scaffold 100 and of providing some or complete remodeling of the prostate. The profiles 133A, B can be sufficient to retain the urethra 14 in an open state through the prostate 10. For example, the lower profile 133 can be smaller than the larger profile 137 but larger than an unexpanded size. In various embodiments the lower profile 133 can be more than about 3 mm, e.g., about 4 mm or more, in some cases about 5 mm, about 6 mm, or more than 6 mm. In one embodiment, the enlarged larger profile 137 is about 9 mm and the enlarged lower profile 133 is about 6 mm. The expanded length 139 can be about the same as the unexpanded length 127 in some embodiments. By minimizing shortening more predictability is provided in the expanded coverage based on the unexpanded position, for example if confirmed by tactile feedback in a delivery system or in visualization. In other embodiments, the expanded length can be shorter than the unexpanded length. Shortening allows the distal and proximal ends 112, 116 to pull back from adjacency to the muscles 26, 30 respectively. This can help assure that the scaffold 100 will not impair the valve function of the muscles 26, 30 contributing to incontinence.

The expanded state of the scaffold 100 illustrated in FIG. 3B is configured to perform well when the central zone 135 is disposed in the area of greatest constriction of the prostate 10, e.g., about in the center of the prostate as measured along the length of the prostate. When so positioned the enlarged central zone 135 focuses the capacity of the scaffold 100 to open the urethra 14 in the zone of greatest impingement.

Although shown as substantially symmetrical about a plane transverse to the longitudinal axis 132 and located midway between the distal end 112 and the proximal end 116, in other embodiments the body 120 is asymmetrical about this plane. An embodiment can be funnel-shaped in which the outer surface 124 tapers inwardly from a central zone toward the distal end 112. This shape enables the elongate body 120 to be lodged in a substantially fixed position in the urethra 14. An example funnel shape can be provided by shortening or eliminating the distal zone 129. This configuration is a proximally oriented funnel, e.g., one with the larger portion facing the bladder 22 and a smaller portion adjacent to the muscle 30. The elongate body 120 is narrowed at the portion closest to the muscle 30. This configuration can induce less dilating effect on the have less dilating effect on the muscle 30 because the structure is tapered toward the distal end 112. Also, the scaffold in this shape can be implanted closer to the muscle 26 which further isolates the muscle 30 from the dilating effect of the scaffold. In some patients it is desirable to induce less dilating effect on the muscle 26. In such cases, a distally oriented funnel shape can be provided. Such a shape has a larger portion facing away from the bladder 22. For example, by shortening or eliminating the distal zone 129 and/or by narrowing the proximal zone 131 the dilating effect of the scaffold on the muscle 26 can be lessened. Also, the scaffold of this configuration may be placed closer to the muscle 30 which further isolates the muscle 26 form the dilating effects of the scaffold.

Another technique for enhancing the chance for preserving continence in a patient is to shorten or eliminate both the distal zone 129 and the proximal zone 131. In this embodiment, the expanded scaffold 100 is tapered from the central plane to the distal end 112 and to the proximal end 116. This approach shortens at least the dimension 139. This approach allows the coverage of the scaffold 100 to be limited to the most constricting central zone of the prostate 10. The distal end 112 can be spaced proximally of the distal end of the prostate 10. The proximal end 116 can be spaced distally of the proximal end of the prostate 10.

FIG. 3C illustrates a portion of one wall pattern 150 that can be incorporated into and/or used to form the scaffold 100. The portion illustrated can correspond to a distal portion, e.g., a distal half, of the elongate body 120. As used in this context, the distal portion is the portion that is closest to the bladder 22 when the scaffold 100 is implanted. In the illustrated embodiment, the first plurality of undulating members 156 is positioned closer to the bladder than the second plurality of undulating members 158. The wall pattern 150 is a flat representation of a distal half of the elongate body 120. The formed scaffold 100 can be provided by connecting upper and lower edges 152, 154 of the wall pattern 150 to form a cylindrical body, e.g., the elongate body 120. In some embodiments a formed cylindrical sheet is provided that is cut to the pattern corresponding to that shown in FIG. 3C. Thus, the flat state illustrated in FIG. 3C is not required. In the illustrated pattern, a first plurality of undulating members 156 and a second plurality of undulating members 158 are provided. The undulating members of the first plurality 156 can have an axial dimension X, e.g., an axial distance between peaks and valleys and can have a circumferential density. Distal apices 156a of the undulating members can be considered the peaks of the pattern. Proximal apices 156b of the undulating members can be considered the valleys of the pattern. The axial dimension X may be referred to herein as an amplitude of the undulating pattern. Elongate members 156c of the undulating members 156 disposed between distal apices 156a and the proximal apices 156b generally exceed the axial dimension X due to being inclined at an acute angle relative to the axial direction (right and left on the page). In the illustrated embodiment the undulating members are shown with relatively sharp angles between adjacent members 156c. FIG. 3D, discussed in more detail below, shows that in other embodiments distal and proximal apices are rounded.

The second plurality of undulating members 158 can have a structure corresponding to that of the first plurality of undulating members 156. In particular, distal and proximal apices 158a, 158b can be provided. The apices 158a, 158b can be connected by elongate members 158c. An axial dimension X can be provided between the distal and proximal apices 158a, 158b. In the illustrated embodiment, the axial dimension X of the first plurality of undulating members 156 is about the same as the axial dimension X of the second plurality of undulating members 158.

In other embodiments, the axial dimension of the second plurality of undulating members 158 differs from the axial dimension of the first plurality of undulating members 156. For example, in a patient with severe prolapse of the urethra more scaffolding effect may be desired to be provided by the second plurality of undulating members 158. This can be accomplished by shortening the dimension X in the zone of the second plurality of undulating members 158 compared to that of the first plurality of undulating members 156. The shortening is one technique to provide a higher axial density of undulating members, measured as circumferential members per unit length in the axial direction (left and right in the page or proximal to distal on the elongate body 120) in the second plurality of undulating members 158 compared to the first plurality of undulating members 156. As discussed more below, the second plurality of undulating members 158 correspond to the portion of the central zone 135 of the scaffold 100.

Circumferential density of the undulating members corresponds to the number of peak, valley, or peaks and valleys. The circumferential density within the first plurality of undulating members 156 of the unexpanded scaffold 100 can be lesser than that of the second plurality of undulating members 158. By enhancing the circumferential density of the undulating members in the second plurality of undulating members 158 the expansion of these members can be greater than that of the first plurality of undulating members 156. In one embodiment, the circumferential density of the second plurality of undulating members 158 can be 50% larger than the circumferential density of the first plurality of undulating members 156. In one embodiment, the circumferential density of the second plurality of undulating members 158 can be 60% larger than the circumferential density of the first plurality of undulating members 156. In one embodiment, the circumferential density of the second plurality of undulating members 158 can be 70% larger than the circumferential density of the first plurality of undulating members 156. In one embodiment, the circumferential density of the second plurality of undulating members 158 can be 80% larger than the circumferential density of the first plurality of undulating members 156. In one embodiment, the circumferential density of the second plurality of undulating members 158 can be 90% larger than the circumferential density of the first plurality of undulating members 156. In one embodiment, the circumferential density of the second plurality of undulating members 158 can be 100% larger than the circumferential density of the first plurality of undulating members 156. In one embodiment, a smooth transition is provided from the circumferential density of the first plurality of undulating members 156 to an enhanced circumferential density in the second plurality of undulating members 158 by gradually increasing number of apices in a direction away from the first plurality of undulating members 156. In some variants, the expansion of the second plurality of undulating members 158 can be increased relative to the expansion of the first plurality of undulating members 156 by lengthening the dimension X in the zone of the second plurality of undulating members compared to that of the first plurality of undulating members 156.

Axial connectors 160 can be provided between longitudinally adjacent undulating members of the first plurality of undulating members 156. Axial connectors 162 can be provided between longitudinally adjacent undulating members of the second plurality of undulating members 158. Axial connector 164 can be disposed between one of the undulating members of the first plurality 156 and one of the undulating members of the second plurality 158. Radial strength and axial flexibility are preferred in some embodiments. As a result, the connectors 160 can be spaced apart by a distance greater than the spacing between circumferentially adjacent proximal apices 156b. In one embodiment, the connectors 160 are at first and second circumferential positions. The first and second circumferential positions can be located such that a plurality of, e.g., two, unconnected proximal apices 156b are disposed therebetween. The connectors 162 can have the same configuration and density as the connectors 160. The connectors 164 can have the same configuration and density as the connectors 160. In other embodiments, the connectors 164 can be longer than the connectors 160 or the connectors 162 by 10%, by 20%, by 40% or by 50%.

The scaffold 100 can be made of any suitable material. In a specific embodiment, the scaffold 100 is bioerodable, which in this context means that the volume of the scaffold 100 is reduced as exposed surface portions are absorbed or dissolved in or otherwise react with urine in the urethra 14 or with the tissue surrounding the urethra. The scaffold 100 is configured to retain its rigidity over time even as its volume is reduced. The analogy is to a piece of hard candy that remains hard as its volume reduces. Eventually the structure is breached to expose more internal areas. The structure can fracture as it erodes to create multiple separated segments. Controlled fracture can be provided by various structure described below, which controlled fracture serves multiple purposes. One such purpose is to provide for self-explantation of small segments as discussed further below.

In certain embodiments the scaffold 100 includes at least one material that is unstable in urine. That is, it can include at least one material that dissolves in or reacts with urine to erode the structure. In certain embodiments the elongate body 120 of the scaffold 100 can include magnesium or a magnesium alloy configured to dissolve when exposed to urine. Other materials that could be used in a dissolving elongate body include zinc and any other biocompatible and bioerodable metal. Polylactic acid based materials could also be used to form the elongate body 120. Also, polylactic-co-glycolic acid could be used in various other embodiments of the elongate body 120. In other embodiments a scaffold is configured for easy retrieval and thus could be made with a biostable material, e.g., one that could reside indefinitely in the urethra.

### III. Structures to Control Treatment Duration

In some embodiments, the elongate body 120 comprises a uniform structure that can slowly react with urine or urethral tissue as discussed above, e.g., by eroding, dissolving, or diffusing into urine or tissue and thereby be extracted from the urethra 14. FIGS. 3D-3F show a number of ways to enhance control of the duration of treatment.

### A. Configurations for Controlled Segmentation and Fracture

FIG. 3D shows a wall pattern 150A similar to the wall pattern 150, except as described differently below. The wall pattern 150A has first and second pluralities of undulating member 156′, 158′. The undulating members have at least some of the proximal and distal apices configured with rounded structures. For example, the undulating member in the first plurality 156′ have distal apices 156a′ and proximal apices 156b′ that are rounded and not angular. For example, the undulating members in the second plurality 158′ also have distal apices and proximal apices that are rounded and not angular. The rounded edges reduce the energy state in the apices which reduce the rate of reaction with the urine or tissue in the urethra 14. The rate of reaction can be focused in other areas. For example the elongate members 156c′ can be configured to more rapidly react, e.g., decay, dissolve, diffuse, or otherwise be eroded in the urethra 14. In one example the elongate members 156c′ can be put under more stress than the apices such that they include a large number of fissures or micro-fractures to expose more material that would otherwise be internal to the flowing urine. In one approach a lesser thickness t2 can be provided in a first part of the first undulating members 156′ and a greater thickness t1 can be provided in another part of the first undulating members 156′. The lesser thickness t2 will be more quickly breached by reaction with urine or tissue in the urethra 14. This will focus fracture at the zone where t2 is provided, causing the elongate body 120 comprising the pattern 150A to be segmented at each portion having the thickness t2.

FIG. 3E illustrates three approaches to configuring the elongate member 156c′ to react more quickly with tissue or urine in the urethra. An elongate member 156c-1 is provided that has a necked down region 170 compared to larger regions 168. The necked down region 170 can have a smaller width, e.g., a dimension transverse to the longitudinal axis of the elongate member 156c-1. The larger regions 168 can include the ends of the elongate member 156c-1 and/or can be contiguous with the apices 156a′, 156b′. The necked down region 170 dissolves before the larger region 168 and/or the apices 156a′, 156b′ because the width in the necked down region 170 is smaller. As a result, the scaffold 100 formed with the structure 156C-1 illustrated by FIGS. 3D-3E will become segmented at the point when the necked down region 170 dissolves. Dissolving in the region 170 can lead to fractures in that region. Such fractures can cause the undulating member to lose radial strength, collapse and be passed out of the body. By providing the region 170, the scaffold 100 will be extracted, e.g., self-explanted, from the patient in pieces that are smaller than a complete undulating member. More particularly, the undulating members when formed into the elongate body 120 form rings that extend circumferentially around the urethra 14 when the scaffold 100 is deployed. When the region 170 fractures, the ring of which it is a part will be segmented into two more arcuate segments. Such segmentation facilitates passing the segmented scaffold out of the urethra 14 with little or lesser discomfort due to smaller size compared to the full intact size of the scaffold 100. Such segmentation also reduces the chance that separated fragments will be lodged on other parts of the scaffold 100 still in place in the urethra.

The necked down region 170 can comprise a reduced thickness of the elongate member 156c in a direction disposed transverse to the longitudinal axis of the elongate member 156c′. More particularly, the necking can be in a thickness direction, as shown in connection with the elongate member 156c-2 in the left image of FIG. 3E. The shaded area corresponding to the necked down region 170 in the image can comprise a section that is reduced in thickness by etching or another process for removing thickness. In some variations, the shaded area corresponding to the necked down region 170 can comprise a large thru-hole extending entirely through the thickness of the member 156C-2. Whether thinned or removed to provide a through hole, the elongate member 156c-2 can dissolve in urine by merely compromising the lateral boundaries of the region 170 or those boundaries and a reduced thickness in the region 170. In the case of a thinned region 170, once the region 170 is breached the remaining portions lateral of the region 170 (to the left and right in FIG. 3E) can more quickly react with urine or tissue in the urethra 14 to breakdown, resulting in controlled fracture.

FIG. 3E shows in connection with the elongate member 156c-3 that the necked down region 170 can include a plurality of smaller reduced thickness areas or perforations. Whether a single region 170 is provided as in the left image or an array of holes is provide as in the right image the amount of area comprising the region 170 can be varied across the length of the scaffold 100 to control not only the location of fracture but the timing of fracture in specific regions of the scaffold 100. Fracture can be pre-programmed by this method to occur first in a proximal region and last in a distal region. Fracture can be pre-programmed by this method to occur in proximal and distal regions before occurring in a central zone.

More particularly, the number of reduced thickness areas or perforations can be varied along the length of the elongate body 120 to control the points of initial fracture. In some cases, the number of reduced thickness areas or perforations can be varied along the length of the elongate body 120 to control the points of initial and subsequent fracture. Referring again to FIG. 3D one notes that the right-most undulating member of the plurality of undulating member 158′ comprises four members 158c′ that are configured to fracture before other elongate member in the undulating member whereas each of the two undulating member disposed distally thereof in the second plurality 158′ comprises three such members. In this embodiment, the right-most undulating member is configured to fracture into four segments. Two undulating member disposed distally of the proximal-most undulating member in FIG. 3D (e.g., on the right hand side in the image) are configured fracture into three segments. The undulating members of the first plurality 156′ are configured fracture into two segments. This demonstrates a technique for controlling the segmentation or fracturing of the elongate body 120 in a preferred manner.

Another manner for controlling the segmenting would be for the proximal-most undulating members to fracture before undulating members disposed distally thereof fracture. Any of the necked down regions 170 can be employed to achieve this result. For example, the scaffold 100 can be configured with a first amount of open or reduced thickness area, e.g., by any of the techniques of FIG. 3E, at or adjacent to the proximal end 116 and can be configured with a second amount of open or reduced thickness area distal to the proximal zone 131, the second amount being less than the first amount. In another embodiment, the amount of open or reduced thickness area in the elongate members 156c-3 a first level in the proximal zone 131, a second level lower than the first level in a portion of the elongate body 120 distal the proximal zone 131, e.g., in the central zone 135. A third level lower than the second level can be provided in another portion of the elongate body 120 distal the proximal zone 131, e.g., in the distal zone 129. This can enable the proximal zone 131 to fracture into segments and be expelled from the urethra before the central zone 135. The central zone 135 can remain intact longer than the proximal zone 131. In one variation the central zone 135 is configured to be expelled after the proximal zone 131 and before the distal zone 129. The distal zone 129 can fracture into segments and be expelled from the urethra generally after the central zone 135. This progressive fracturing from proximal-to-distal helps minimize the risk of having segments from upstream zones of the scaffold 100 become lodge in downstream portions of the scaffold, which could lead to a constriction of flow in the urethra 14.

In another embodiment, a focus is on reducing the impact on the distal and proximal muscles 26, 30. As such, a technique can be followed for providing a necked down region 170, e.g., as shown in any of the embodiments of FIG. 3E, yielding an enhanced amount of open or reduced thickness area in the elongate members 156c at or adjacent to the distal end 112 and at or adjacent to the proximal end 116. The central zone 135 can have less or no necked down regions 170. As an example, the elongate members 156c-3 can have an amount of open or reduced thickness area at a first level in the central zone 135 and can be at a second level higher than the first level in the proximal zone 131. The reduced thickness area in the elongate members 156c-3 can be higher in the distal zone 129 than in the central zone 135. This can enable the distal and proximal zones 129, 131 to fracture into segments and be expelled from the urethra while leaving the central zone 135 intact in the prostate 10 keeping the urethra 14 open in the zone that was constricted prior to treatment while providing more space between the proximal-most end of the elongate body 120 and the muscle 30 and between the distal-most end of the elongate body 120 and the muscle 26. Such space can increase the chance for the patient to avoid incontinence while at the same time maintaining normal flow through the region of the urethra 14 flowing through the prostate 10.

### B. Configurations for Extended Treatment

In certain embodiments the elongate body 120 includes a material that is eroded by urine and/or absorbed in the tissue surrounding the urethra 14 and the material is surrounded by a layer that can delay such erosion and/or absorption. FIG. 3F shows a scaffold 100D is provided that includes more than one layer. In one embodiment at least some filaments 178 of the elongate body 120 include an inner portion 180 and an outer portion 184. The inner portion 180 can comprise a material that dissolves or otherwise is eroded from the rest of the elongate body 120 when exposed to urine. The inner portion 180 can include any of the materials described herein including any of magnesium, a magnesium alloy, zinc, PLLA, PLGA or another material as described above. The outer portion 184 can be a layer of material that differs from the inner portion 180 in response to exposure to urine, for example being slower to dissolve in urine or being stable without substantial erosion or dissolution in the presence of urine.

The outer portion 184 can be a coating or other layer that is applied by any suitable technique. The outer portion 184 can be configured to modulate the erosion process. The coating can be polymer that initially completely encapsulates the inner portion 180 of the scaffold 100 to prevent it from contacting the urine. The coating can be configured to be biodegradable, such that over a period, e.g., over thirty days, it gradually exposes all or portions of the scaffold to permit the erosion process to commence. The coating need not retain rigidity and can more rapidly degrade than the scaffold 100.

The outer portion 184 can be a coating that is loaded with an agent, e.g., a drug, to treat the prostate 10 to reduce the size thereof or to eliminate re-growth or re-encroachment of the prostate 10 into the passage of the urethra 14 or to enhance healing after a urinary procedure. Suitable agents to include in or as the outer portion 184 include an antibiotic, an anti-coagulant, an agent including heparin, one or more endothelialization factors, such as antibodies for deposition of endothelial progenitor cells, and other beneficial cell growth media. Other agents that can be used as or in the outer portion 184 can include collagen, dextran or other sugar substance, plasma, or a biocompatible wax. These agents may over time be liberated from the scaffold 100 exposing the inner portion 180.

The outer portion 184 can include a material that while being slower to dissolve than the inner portion 180 or being inert to urine can be pierced, severed or otherwise rapidly removed under select conditions to expose the inner portion 180. In some embodiments, the outer portion 184 can be a material that dissolves at a slow rate compared to the inner portion 180. The outer portion 184 can be a material that dissolves at the same rate as the inner portion. The thickness of the outer portion 180 can be sufficient to delay the exposure of the inner portion 180 to urine by a time sufficient to provide remodeling, reduction in swelling or other result that causes the urethra 14 to no longer be obstructed by the prostate 10.

In one embodiment, the outer portion 184 includes a coating disposed around the inner portion 180, which is formed of magnesium or a magnesium alloy. The coating is configured to prevent exposure of the magnesium or magnesium alloy to urine for a minimum of thirty days when immersed in urine. In other embodiments, the coating is configured to prevent exposure of the magnesium or magnesium alloy to urine for a minimum of sixty days when immersed in urine. In other embodiments, the coating is configured to prevent exposure of the magnesium or magnesium alloy to urine for a minimum of one hundred and twenty days when immersed in urine. In other embodiments, the coating is configured to prevent exposure of the magnesium or magnesium alloy to urine for a minimum of one hundred and eighty days when immersed in urine. In other embodiments, the coating is configured to prevent exposure of the magnesium or magnesium alloy to urine for a minimum of one year days when immersed in urine. The coating can be completely inert.

As discussed further below, the coating can be configured to be pierced to expose the magnesium layer in some embodiments. The coating can be pierced by a sharp implement pressed into the inner surface 128D. The coating can be pierced by a pressure that is uniformly applied to the inner surface 128D.

The outer layer 184 can completely encapsulate the inner portion 180 as shown. This can isolate the inner portion 180 from absorption into the tissue around the urethra 14 as well as erosion into the urine. The outer layer 184 can be or can be disposed over only an inner surface 128D of the elongate body 120D. While the outer surface 124 can be absorbed into the tissue, this process may be much slower than the reaction with the urine. The outer surface 124 is generally not in contact with urine because it is generally buried in the tissue of the urethra 14 and thus not persistently exposed to urine when the scaffold 100 is implanted.

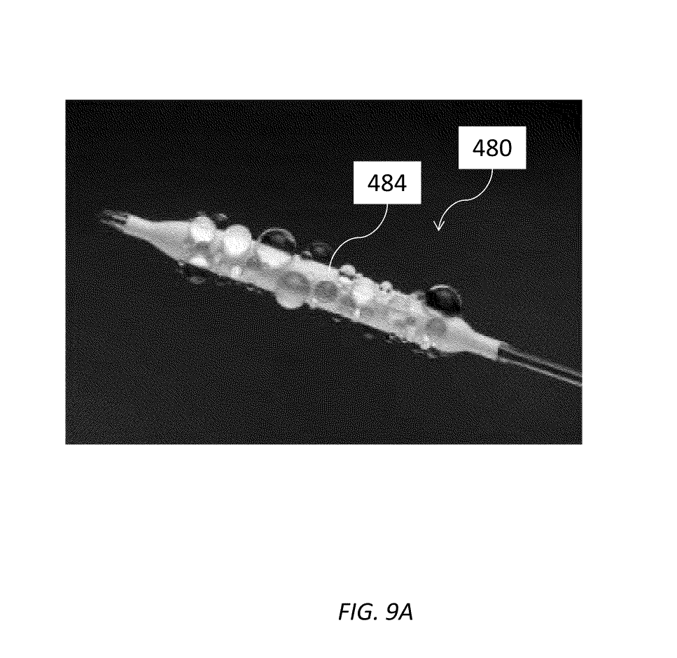

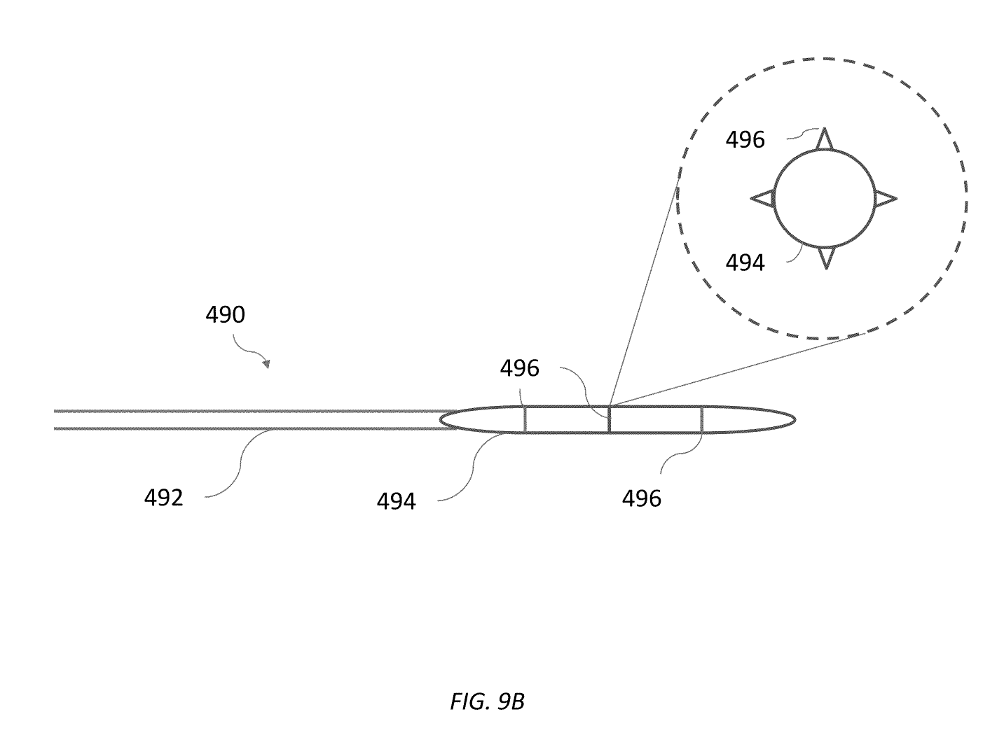

In one embodiment, the elongate body 120 is configured to remain intact for at least thirty days to provide a therapy. If the outer portion 184 is not present, the elongate body 120 may begin to erode in the urine immediately but has sufficient thickness or sufficiently slow rate of erosion that the structural integrity of the scaffold 100 is not depleted until after thirty days. If the outer portion 184 is present, the outer portion 184 may be configured to not be breached by urine before a treatment period such as before thirty days. In some approaches, the outer portion 184 is breached by the outer portion 184 eroding in urine or by an external means, such as by being scored by another device as discussed below in connection with FIG. 9B.

### IV. Delivery Systems & Related Devices

FIGS. 4-8 illustrate various embodiments of delivery systems that can be used to place the scaffold 100. The system 400 can include an anchor catheter 404 and a scaffold deployment catheter 408. The deployment catheter 408 is sometimes referred to herein as a delivery catheter. The anchor catheter 404 can also serve as a guide structure for the scaffold deployment catheter 408. The anchor catheter 404 has sufficient stiffness to push through the urethra but has a soft, blunt distal end such that interactions with the inside of the bladder are atraumatic. The scaffold delivery catheter 408 is configured to couple with the scaffold 100 to deliver the scaffold to the region of the urethra 10 about which the prostate 14 is located. FIG. 4 shows that the scaffold delivery catheter 408 can be coaxially mounted around the anchor catheter 404.

The system 400 can have an overall length sufficient to reach from outside the penis, through the urethra 14 and into the bladder 22. The system 400 also has length sufficient to accommodate two or more handles which will be located outside the patient in a procedure. The length of the system 400 from proximal end of the anchor catheter 400 to the distal end thereof can be about 1150 mm (about 45 inches).

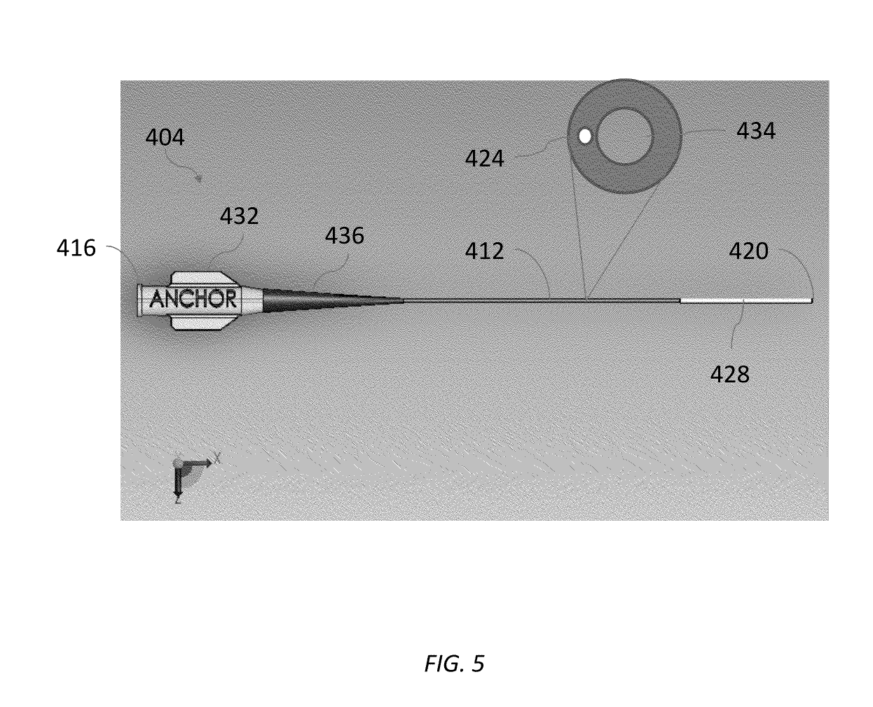

FIG. 5 shows that the anchor catheter 404 can include an elongate body 412. The elongate body 412 has a proximal end 416, a distal end 420 and a lumen 424 disposed between the proximal and distal ends. In the illustrated embodiment an anchor balloon 428 is coupled with the elongate body 412 at or adjacent to the distal end 420. In one embodiment the lumen 424 is configured to deliver an inflation medium to the anchor balloon 428. The anchor balloon 428 is disposed on the distal end 420 in one embodiment with the anchor balloon 428 in fluid communication with the lumen 424. In variations, the anchor balloon 428 can be configured as a mechanical device that expands without being inflated, e.g., as a mesh that can be expand by shifting a distal end of the mesh toward a proximal end of the mesh. Other mechanically expandable anchors can be used.

FIG. 5 shows that the proximal end 416 of the elongate body 412 has a connection hub 432 disposed thereon. A strain relief 436 is disposed between the connection hub 432 and the elongate body 412. The strain relief 436 is tapered as illustrated. The hub 432 can be configured to connect a source of inflation media (not shown) with the lumen 424. The hub 432 can be configured to connect a urine drainage lumen 434 with a waste receptacle.

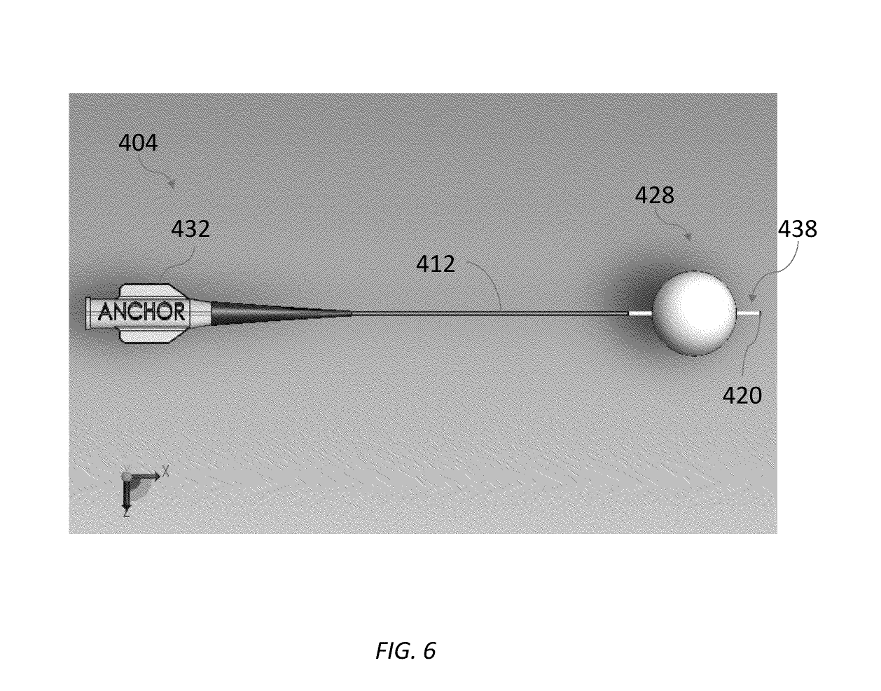

FIG. 6 shows the anchor balloon 428 in an expanded configuration. The anchor balloon 428 has a proximal face configured to be coupled with an interior of a bladder 22 and to retain the anchor catheter 404 in the bladder. Once anchored the elongate body 412 remains in place and provides a traction member for advancing the scaffold delivery catheter 408. In one embodiment the anchor balloon 428 is spaced from the distal end 420. A soft, blunt tubular body 438 disposed between the distal end 420 and the anchor balloon 428 assures atraumatic interaction with the bladder 22. The tubular body 438 spaces the distal face of the anchor balloon 428 from the wall of the bladder 22 opposite the opening 18 to the urethra 14. Although shown as a straight segment, the tubular body 438 can be arcuate such as a pig-tail shape. In some embodiments, the tubular body 438 is compressible or floppy such that it can be deformed upon engagement with an inside surface of the bladder 22 rather than piercing the tissue around the bladder 22.

Generally, the anchor catheter 404 is configured to be advanceable through the urethra and in particular through a constricted portion thereof. For example, the elongate body 412 can be sufficiently pushable by having a diameter of about 0.7 mm. The balloon 428 can be configured to be retained in the bladder 22 by being larger than the opening 18 but preferably without filling the entire volume of the bladder. The balloon 428 can have a radius when expanded of about 9.5 mm.

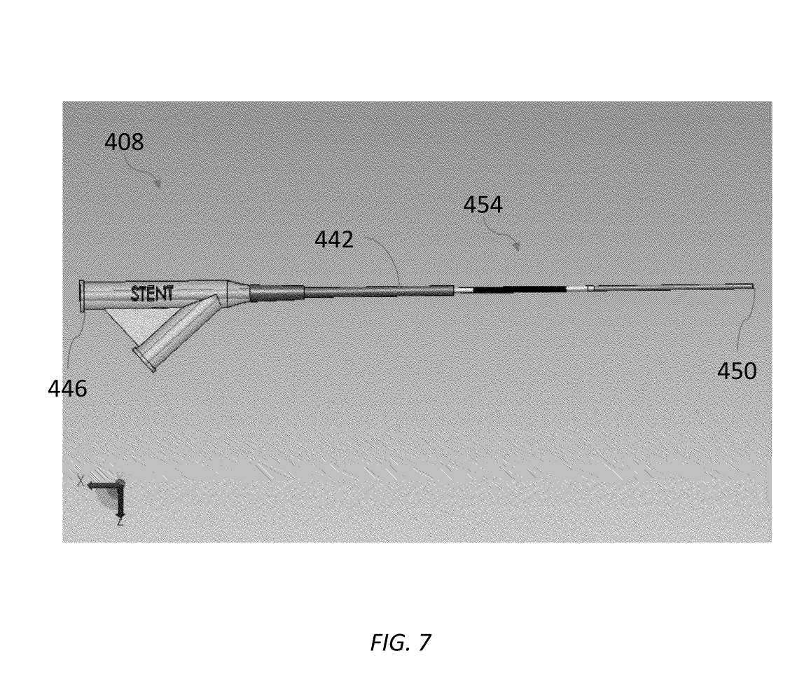

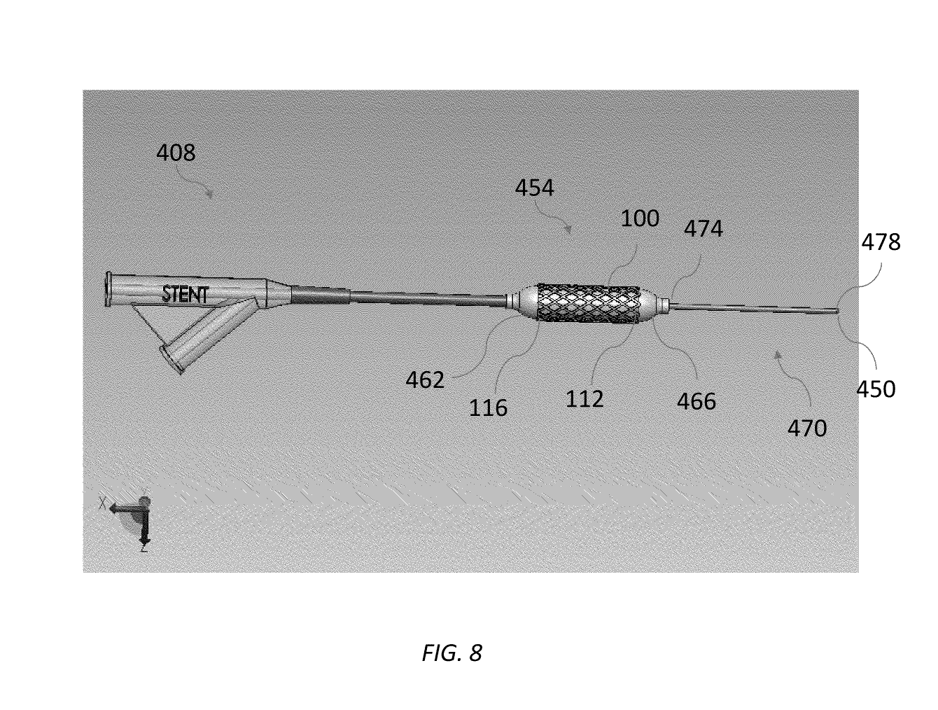

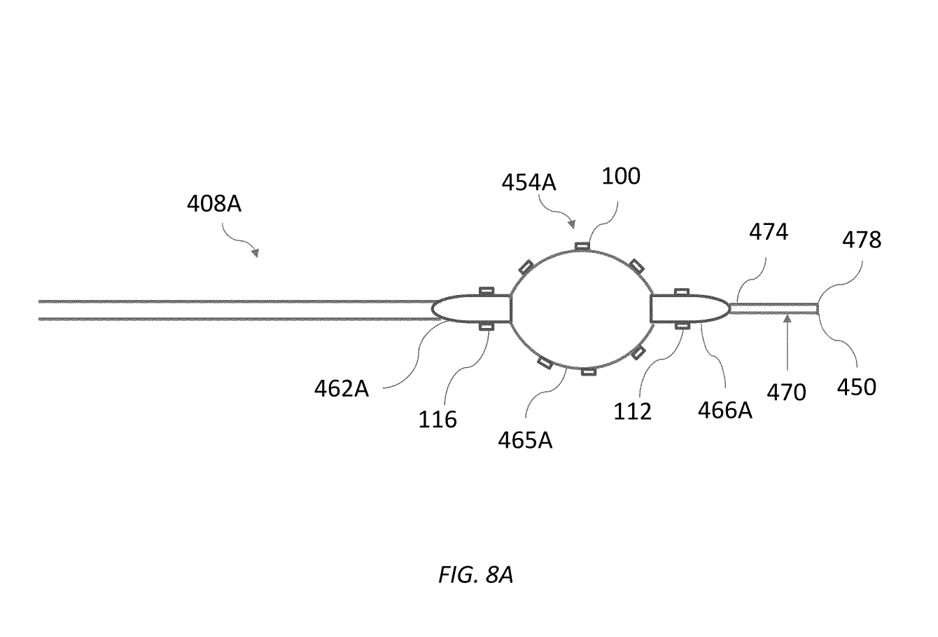

FIGS. 7 and 8 show an embodiment of the scaffold delivery catheter 408. The delivery catheter 408 is an assembly that includes an elongate body 442. The elongate body 442 includes a proximal end 446, a distal end 450 and a lumen (not shown) disposed between the proximal and distal ends 446, 450. The lumen can be centrally disposed in the elongate body 442. The lumen can be configured to receive the elongate body 442 of the anchor catheter 404 as shown in FIG. 4. The delivery catheter 408 has an expandable member 454 disposed on a side surface of the elongate body 442. The expandable member 454 can be a balloon or an expandable member that is expanded mechanically by other means. The expandable member 454 is located adjacent to but spaced from the distal end 450. The expandable member 454 can be in fluid communication with an inflation lumen (not shown). The inflation lumen can be separate from a tracking lumen that is configured to receive the elongate body 412. For instance the inflation lumen can be disposed peripherally of the tracking lumen. The inflation lumen can be much smaller than the tracking lumen configured to receive the elongate body 412.

The expandable member 454 comprises a proximal end 462, a distal end 466, and a length disposed therebetween. The length between the proximal and distal ends 462, 466 is sufficient to mount the scaffold 100 thereon. A proximal end 116 of the scaffold 100 is disposed distally of the proximal end 462 of the expandable member 454. A distal end 112 of the scaffold 100 is positioned proximally of the distal end 466 of the expandable member 454. The profile, e.g., the shape as viewed from the side, of the expandable member 454 between the proximal and distal ends 462, 466 can have any configuration. In one embodiment, the profile corresponds to a cylindrical shape such that the scaffold 100 can be generally uniformly expanded over its length. In one embodiment, the profile of the expandable member 454 corresponds to a varying size profile. The profile can include non-linear, e.g., arcuate or convex, segments along the length between the proximal and distal ends 462, 466. The size of the expandable member 454 can be larger at locations proximal of the distal end 466. The size of the expandable member 454 can be larger at locations distal of the proximal end 462. The size of the expandable member 454 can be larger at locations between the proximal and distal ends 462, 466.

FIG. 8A shows a distal portion of an embodiment of the delivery catheter 408A in which an expandable member 454A can be configured to expand to a shape matching the expanded shape of the scaffold 100 as shown in FIG. 3B. The expandable member 454A has a distal portion 466A that is generally constant diameter along a distal portion including the distal end 112 of the scaffold 100. The expandable member 454A has a proximal portion 462A that is generally constant diameter along a proximal portion including the proximal end 116 of the scaffold 100. A central portion 465A of the expandable member 454A increases in diameter from the proximal portion 462A to a mid-portion of the central portion 465A and then decreases from the mid-portion to the distal portion 466A.

The system 400 enables careful placement of the scaffold 100 in the urethra 14. The placement is aided in one embodiment by a distal projection 470 on the deployment catheter 408, 408A. The projection has a proximal end 474 disposed adjacent to the distal end 466 of the expandable member 454 and a distal end 478 disposed away from the proximal end 466. The distal end 478 can be disposed at the distal-most end of the delivery catheter 408, 408A. The length of the distal projection 470 can be sufficient to place the scaffold 100 within the region of the prostate 10 as discussed further below. In one embodiment the system 400 is configured such that when the distal end 478 touches the proximal face of the anchor balloon 428 the distal projection 470 extends from the opening 18 through the distal sphincter muscle 26 and to a location proximal of the distal end of the prostate. In use, the proximal end 474 of the distal projection 470 is located just distal of the distal end of the prostate 10 such that the distal end 112 of the scaffold 100 also is close to, but proximal of the distal end of the prostate. In use, the proximal end 474 of the distal projection 470 is located just distal of the distal end of the prostate 10 such that the proximal end 116 of the scaffold 100 also is close to, but distal of the proximal end of the prostate. The distal projection 470 assures that the central zone of the scaffold 100 is located within the central zone of the prostate 10. The distal projection 470 helps retain the scaffold 100 within the prostate 10 without causing the distal end 112 to encroach into the distal sphincter 26 and without causing the proximal end 116 to encroach into the proximal sphincter 30.

FIG. 4 and FIGS. 5-8 show embodiments in which two catheter bodies can be slideable over each other to facilitate delivery of the scaffold 100. In some embodiments this feature is eliminated. FIG. 4A shows an embodiment of a system 400A in which a balloon 428A is provided for anchoring a delivery catheter 408A in the urethra 14. The system 400A can be delivered such that the balloon 428A is disposed in the bladder 22. The balloon 428A is then expanded. The expandable member 454 is spaced proximally from the anchor balloon 428A by a fixed amount. The fixed amount is that corresponding to the distance between the neck of the bladder 22 and the location of prostate 10. For example, in one method the distance from the neck of the bladder 22 to the prostate 10 is confirmed by ultrasound or other imaging technique. Thereafter, an appropriately sized system 400A is selected, e.g., one having the proper distance between the balloon 428A and the balloon 454. In some variants, the anchor balloon 428A and the balloon 454 for expanding the scaffold 100 can be inflated using the same inflation lumen. For example, the anchor balloon 428A can be inflated at a first inflation pressure (e.g., 2 ATM). The first inflation pressure can be insufficient to inflate the balloon 454. The proximal face of the inflated anchor balloon 428A can be positioned against an interior surface of the bladder, thereby aligning the balloon 454 at the desired position within the urethra. The inflation pressure within the inflation lumen can then be increased to a second inflation pressure (e.g., 5 to 10 ATM). The second inflation pressure can be sufficient to inflate the balloon 454, thereby causing the balloon 454 to expand the scaffold 100. A configuration that facilitates a shared inflation lumen provides that the anchor balloon 428A is formed of a non-compliant or a generally low compliance balloon material. As such, the balloon 428A expands to a pre-defined size. Or the balloon 428A expands relatively rapidly to a first size and then expands at a much lower rate thereafter such that the inflation medium instead flows into the balloon 454 to expand the scaffold 100. In one embodiment, the system 400A is configured to minimize the impact on at least one of the two sphincter muscles 26, 30. The distance from the balloon 428A to the distal end of the scaffold 100 can be confirmed to be greater than the distance from the neck of the bladder 22 to the distal sphincter muscle 26. This approach can protect the muscle 26 against being dilated by a distal portion of the scaffold 100, reducing the likelihood of incontinence through the distal muscle 26. The distance from the balloon 428A to the proximal end of the scaffold 100 can be confirmed to be less than the distance from the neck of the bladder 22 to the proximal sphincter muscle 30. This approach can protect the muscle 30 against being dilated by a proximal portion of the scaffold 100, reducing the likelihood of incontinence through the proximal muscle 30. The distance from the balloon 428A to the proximal end of the scaffold 100 can be confirmed to be less than the distance from the neck of the bladder 22 to the proximal sphincter muscle 30 and the distance from the balloon 428A to the distal end of the scaffold 100 can be confirmed to be greater than the distance from the neck of the bladder 22 to the distal sphincter muscle 26. This approach can protect the muscle 30 and the muscle 26 against being dilated by the scaffold 100, reducing the likelihood of incontinence through both of these muscles.

FIG. 4B shows a system 400B that is a variation on the system 400A. The system 400B can have any of the features of the system 400A and can be used in the same ways. In the system 400B, a three channel hub 504 is provided. The three channel hub 504 provides a first channel 508 for inflating the anchor balloon 428A. A second channel 512 is provided for inflating the balloon 454. A third channel 516 is provided for providing fluid communication from the bladder 22 to the outside of the patient. The third channel 516 allows the bladder to be drained during the procedure or during recovery if necessary. This would allow the delivery catheter 408B to be left in place after the procedure for at least a brief recovery period. In some variants, the first channel 508 can be used for inflating the anchor balloon 428A and the balloon 454, as described above with regard to an embodiment of the system 400A. The second channel 512 and the third channel 516 can provide fluid communication from the bladder 22 to the outside of the patient. The second and third channels 512, 516 can be used to irrigate the bladder 22 before, after, or during implantation of the scaffold 100. For example, a fluid (e.g., saline) can be introduced into the bladder 22 through the second channel 512, and the contents of the bladder 22 can be aspirated or drained through the third channel 516, thereby allowing the bladder 22 to be irrigated or flushed out. In some variants, the bladder 22 can be irrigated in order to remove blood clots from the bladder 22. In some embodiments, the bladder 22 can be irrigated using one channel alone (e.g., the third channel 516). A fluid (e.g., saline) can be introduced into the bladder 22 through the third channel 516. The bladder 22 can become pressurized during the inflow of the fluid. The bladder 22 can then be drained through the third channel 516, thereby relieving the pressure within the bladder 22.

FIG. 4C illustrates a delivery system 400C in which no anchor balloon is provided. The system 400C is similar to the system 400 except as described differently below. The system 400C includes a delivery catheter 408C that is configured to place the scaffold 100. The system includes a distal portion including the expandable member 454. The system 400C also includes a first marker 520 and a second marker 524. The first and second markers 520, 524 can be integrated into the body of the catheter at or adjacent to the balloon 454. The first marker 520 can be placed to give an indication of where the location of the scaffold 100 relative to the muscle 30. The second marker 524 can be placed to give an indication of where the location of the scaffold 100 relative to the muscle 26. In some cases just one of the markers is provided. The delivery catheter 408C can have the distal projection 470, which can extend into the bladder 22 even when the balloon 454 has been properly positioned at the prostate 10.

FIG. 9A illustrates a treatment catheter 480 and includes an expandable member 484 that can be used in place of any of expandable members 428, 454, 454A of the system 400 illustrated in FIG. 4. The catheter 480 can have the same or similar features as the catheter 404 or the catheter 408. In some variations, the catheter 480 also has an expandable member 484 that is configured to permit the inflation medium or a substance included with the inflation medium to seep through the wall of the inflation medium. For example, the expandable member 484 can have a weeping balloon configuration where a liquid delivered into the balloon can escape through the wall of the balloon. As discussed above, the anchor catheter 404 can be configured with a lumen to permit urine to pass therethrough. As such the obstructed effect of expanding the member 484 on the catheter 480 will not prevent emptying of the bladder. The expandable member 484 can thus remain inflated within the space of the prostate for as long as needed to provide a treatment of the prostate.

In certain methods, the expandable member 484 is used to deliver the scaffold 100. The catheter 480 can be used in other methods separate from the scaffold 100. That is, the catheter 480 can be used to deliver a therapeutic agent for a targeted therapy to the prostate 10. Therapeutic agents of interest are generally fast absorbing agents that can be passed through the catheter 480. Such agents can include an antibiotic, a cell inhibitor such as paclitaxel, a vitamin that can be eluted from or coated on the expandable member 484.

In another method, the catheter 480 can be delivered after the scaffold 100 is in place. For example if it is desired to remove the scaffold 100, the catheter 480 can be advanced into the urethra 14 and expanded and a medium that accelerates the dissolving of the scaffold 100 can be eluted from the balloon 484. For example, if the scaffold is coated as discussed above in connection with FIG. 3F, the balloon 484 could be inflated with a medium that quickly compromises the outer portion or layer 184 exposing the inner portion 180. In one technique, a mild acid can be used to pressurize the balloon 484 and the acid can seep out of the balloon 484 to etch the material. The acid can be selected to create necked down regions similar to the regions 170 and in any pattern as discussed above. The acid can be selected to remove the coating 184 to expose the inner portion 180 to urine to enhance breakdown of the scaffold 100.

FIG. 9B shows a catheter 490 that can be used to expedite removal of the scaffold 100. The catheter 490 has an elongate body 492 that can extend from outside the penis through the urethra 14 and into the region of the prostate where the scaffold has been deployed. The catheter 490 can be delivered over the anchor catheter 404, over a guidewire or unguided. The catheter 490 can include an expandable member 494, such as a balloon. The balloon can include one or a plurality of scoring features 496 disposed on the expandable member 494. The catheter 490 is shown as having three scoring features 496. A scoring feature 496 can be provided for one or more of the distal zone 129, the proximal zone 131, and the central zone 135. In one embodiment the scoring feature 496 can be provided in each of the distal zone 129, the proximal zone 131, and the central zone 135. The expandable member 494 can have a scoring features 496 corresponding to each circumferential member. Fewer scoring features 496 can be provided, for example limiting the scoring features to locations corresponding to the distal zone 129 or the proximal zone 131. The inset image shows a cross-section of the expandable member 494 and the scoring features 496. The scoring features 496 are shown schematically as having a wide base and a pointed tip. More generally, the scoring features 496 should be configured such that upon expansion of the expandable member 494, the scoring features 496 create one or more breaches in a coating or cracks in a surface of a portion of a circumferential member or connector to cause the scaffold 100 to fracture or be weakened or to induce a high energy state where dissolving of the scaffold is accelerated. The scoring features 496 can be configured to minimize damage to the surrounding tissue. For example, the height of the scoring feature 496 can be selected so that the scoring feature 496 extends beyond the expandable member 494 a radial distance that is less than the thickness of a strut of the scaffold 100. In some embodiments, the scoring feature 496 can extend a sufficient longitudinal distance so that the scoring feature 496 cannot escape the cage-like structure of the scaffold 100. For example, the scoring feature 496 can extend from the proximal-most end of the scaffold 100 to the distal-most end of the scaffold 100. In some variants, the scoring feature 496 can extend a longitudinal distance that is sufficient to span across two, or three, or four, or more circumferential rings of the undulating members 156, 158.

The catheter 490 can be used to expedite explantation of the entire scaffold 100. In one variation, the catheter 490 is used to modify an implanted scaffold to expedite explant of only a portion of the scaffold 100. For example, the central scoring feature 496 can be eliminated such that expansion of the expandable member 494 causes the scoring features to score the distal zone 129 and the proximal zone 131. This causes distal zone 129 and the proximal zone 131 to be explanted after urine erodes the scored sections such that these portions are removed. This leaves the central zone 135 in place. In another embodiment, the distal scoring feature 496 is provided, but the rest or the balloon 494 is smooth. This allows a clinician to remove the distal zone 129 leaving the central zone 135 and the proximal zone 131 in place. By removing the distal zone 129 the dilation of the distal sphincter muscle 26 can be reduced. This can help to prevent incontinence of the distal muscle 26. In another embodiment, the proximal scoring feature 496 is provided, but the rest or the balloon 494 is smooth. This allows a clinician to remove the proximal zone 131 leaving the central zone 135 and the distal zone 129 in place. By removing the proximal zone 131 the dilation of the proximal sphincter muscle 30 can be reduced. This can help to prevent incontinence of the proximal muscle 30.

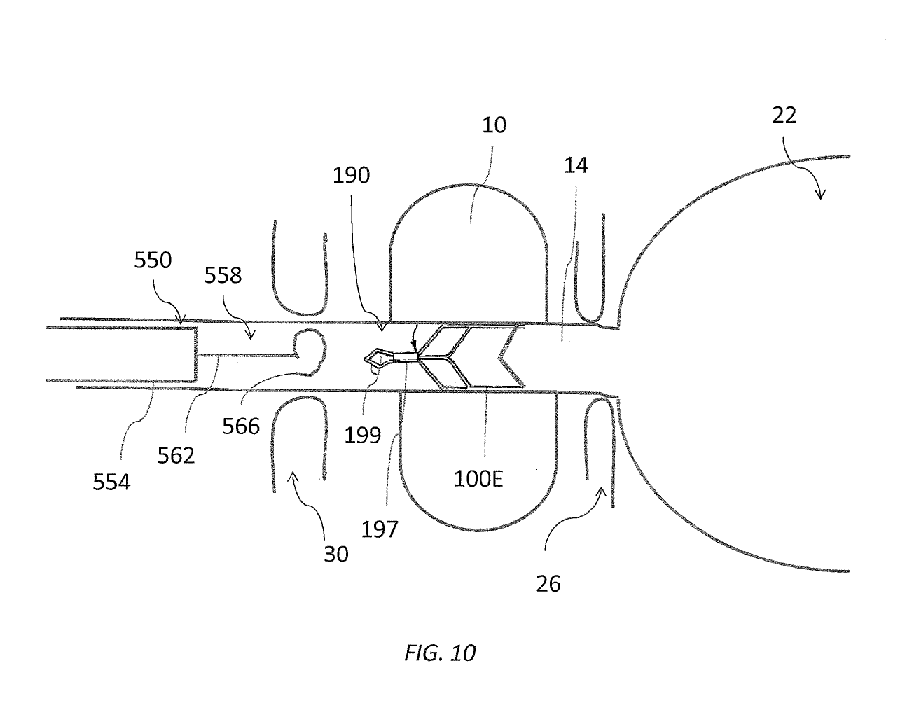

FIG. 10 shows another embodiment an assembly a scaffold 100E and a snare 190 to facilitate extraction. The scaffold 100E is can be balloon expandable but in the illustrated embodiment is self-expanding by having a plurality of peripheral axial struts that can expand into apposition with the urethra 14 in the zone of the prostate 10 to open up a blockage. The axial struts are braced by a plurality of angled members that provide for expansion and compression. The snare 190 can be coupled with a proximal most portion of the scaffold 100E, e.g., with a plurality of angled members thereof. The snare 190 includes a hub 197 and a snare feature 199 coupled with an end of the hub 197 opposite of the end to which the scaffold 100E is coupled. The configuration of the scaffold 100E and the hub 197 is such that the snare feature 199 can be a loop that is suspended in the urethra 14 spaced from the wall thereof.

### Methods of Delivery & Bail Out

As discussed above, this application is directed to positioning a scaffold within the urethra 14 in the portion thereof surrounded by the prostate to provide relief for narrowing of the urethra due to enlargement of the prostate for whatever reason. The scaffolds disclosed herein are preferably configured for temporary placement. The scaffold can be made temporary by configuring it to be removed or to be self-removing over time.

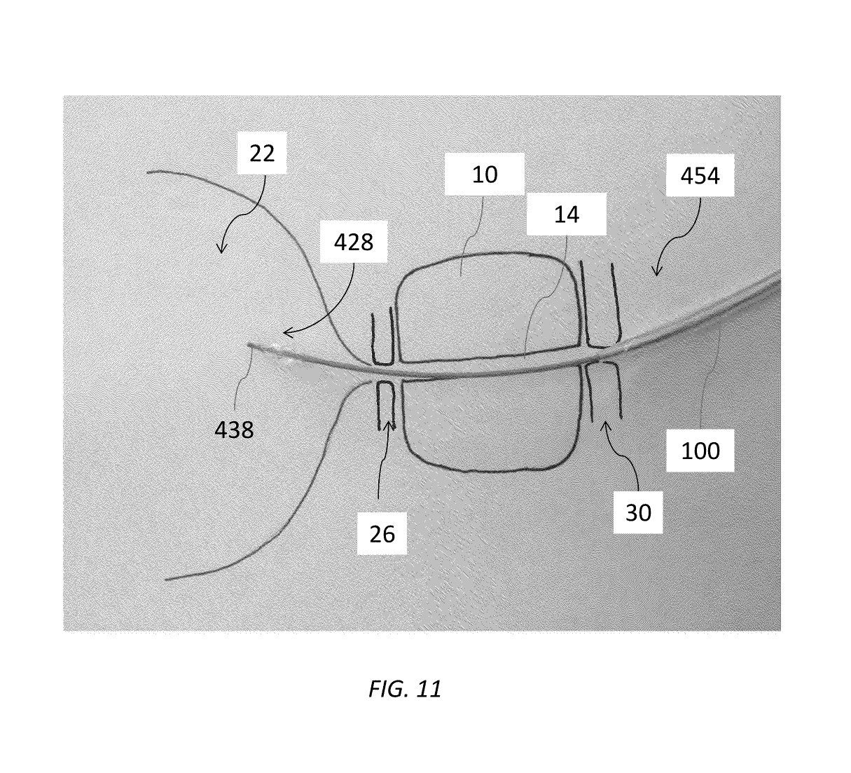

FIGS. 11-15 illustrate methods of using the system 400 for treating a patient with an enlarged prostate. FIG. 11 shows that the anchor catheter 404 is delivered into the urethra and advanced until the anchor balloon 428 is disposed within the bladder 22. The soft blunt tubular body 438 can be used to provide tactile feedback of the position of the anchor catheter 404 before the balloon 428 is expanded. Or the position of the anchor catheter 404 can be confirmed by imaging technology such as fluoroscopy. Thereafter the anchor balloon 428 is inflated. The anchor balloon 428 can be inflated in the free volume of the bladder 22 and thereafter positioned in the bladder to anchor the anchor catheter 404. For example, the anchor balloon 428 can be inflated and then the elongate body 412 can be put in tension or translated proximally to bring the anchor balloon 428 into contact with the bladder 22 adjacent to the opening into the urethra 14. FIG. 11 schematically shows the prostate open with a portion of the body of the anchor catheter 404 disposed therein. In some cases, the prostate is much more prolapsed into the urethra 14 creating difficulty in passing the catheter 404 therethrough. The soft blunt tubular body 438 provides for passage through such obstructed portions of the urethra 14.

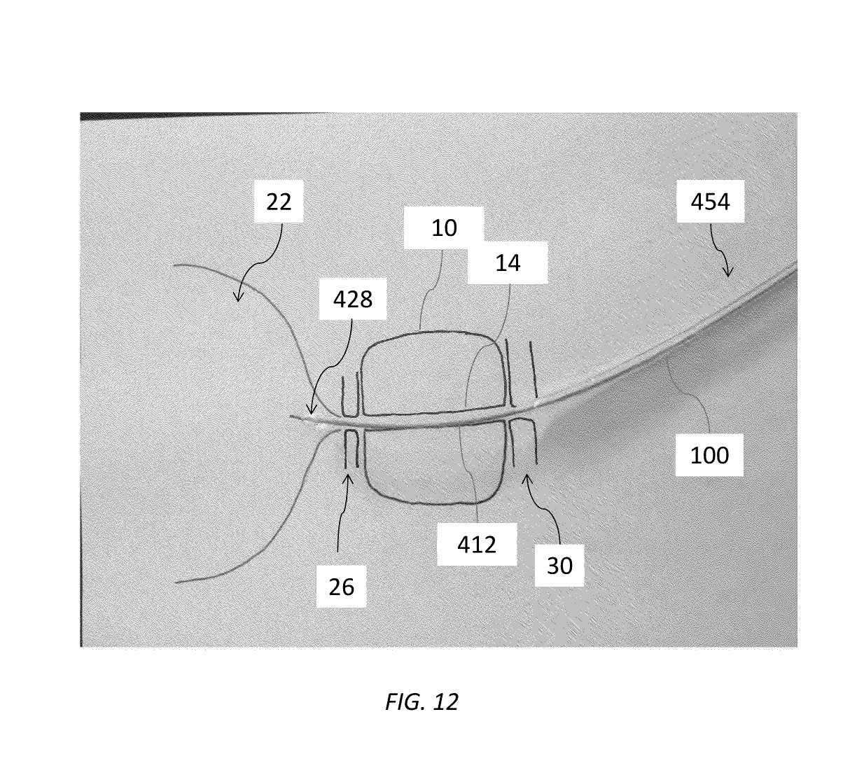

FIG. 12 shows that the anchor balloon 428 engage the tissue around the opening 18 from the bladder 22 into the urethra 14. Once so anchored, the elongate body 412 of the anchor catheter 404 can be used to guide the delivery catheter 408 into place in the urethra 14 in embodiment where the delivery catheter 408 and the anchor catheter 404 are telescoping or otherwise moveable relative to each other. As shown, the delivery catheter 408 can be advanced along the elongate body 412 until the scaffold 100 is positioned in the region of the urethra 14 just proximal of the prostate 10. The delivery catheter 408 can be positioned such that the scaffold 100 is just proximal of (downstream of) the muscle 30.

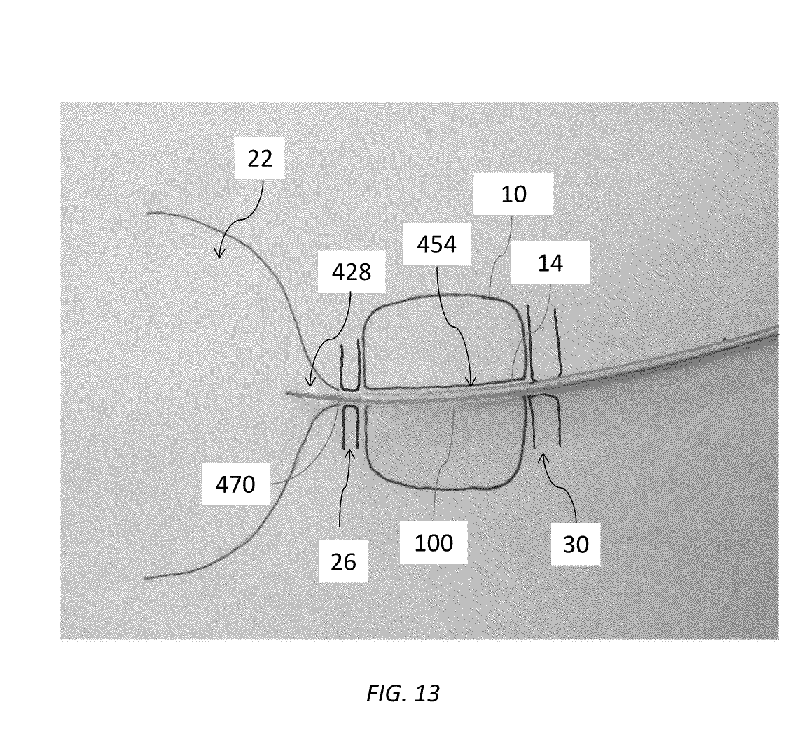

FIG. 13 shows the delivery catheter 408 advanced until the scaffold 100 is within the length of the urethra 14 surrounded by the prostate 10. For example, the proximal end 112 of the scaffold 100 can be positioned just distal the proximal end of the prostate 10 and the distal end 116 of the scaffold 100 can be positioned just proximal the distal end of the prostate 10. Positioning of the scaffold 100 within the prostate in this manner can be aided by tactile feedback. For example, the distal projection 470 can be advanced until it abuts the balloon 428 or a member or stub just proximal of the balloon. In other techniques, ultrasound or other imaging techniques can be used to assure proper placement of the scaffold 100. In certain embodiments, the location of the distal end 112 of the scaffold 100 being proximal of the muscle 26 is confirmed prior to expansion of the balloon 454. In certain embodiments, the location of the proximal end 116 of the scaffold 100 being distal to the muscle 30 is confirmed prior to expansion of the balloon 454. This confirmation of placement can be done by tactile feedback and some knowledge of the specific patient's anatomy or by visualization. If the delivery system provides for relative motion of the catheter 408 over the catheter 404, then the balloon 428 can be pulled back into engagement with the neck of the bladder 22 and while holding the catheter 404 in this manner, the position of the catheter 408 can be adjusted as appropriate.

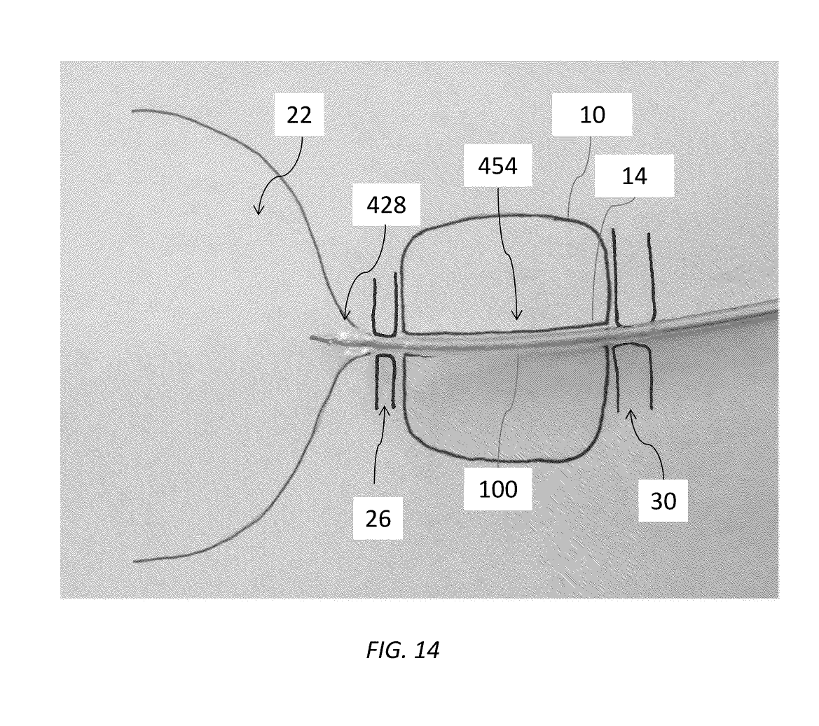

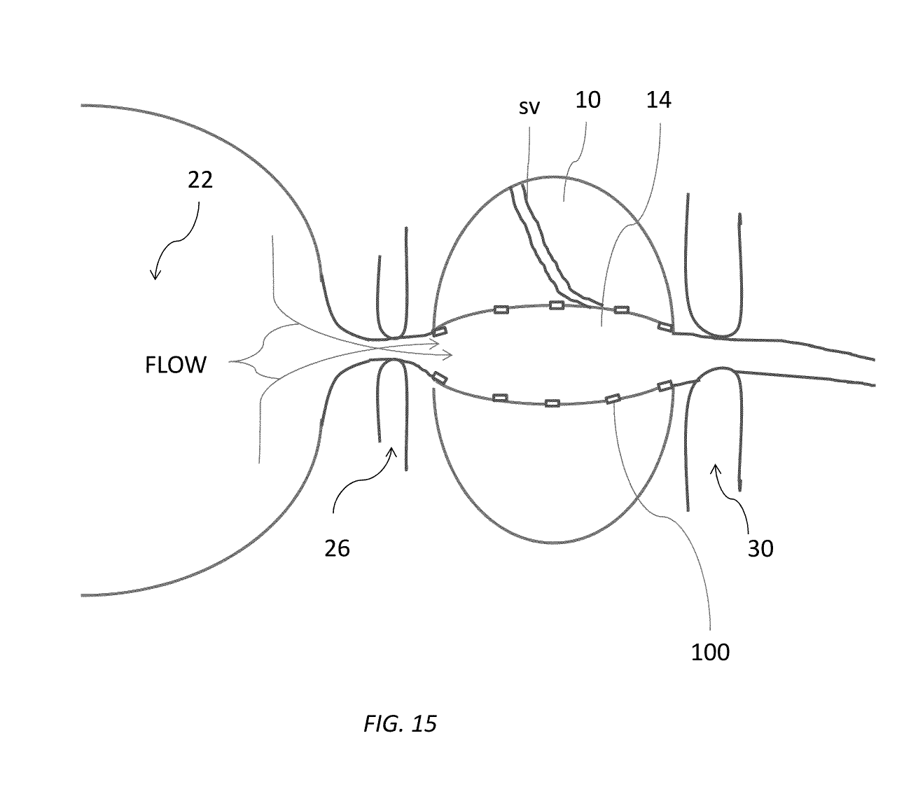

FIG. 14 shows that thereafter the expandable member 454 can be expanded causing the scaffold 100 to be expanded into engagement with the inside wall of the urethra 14. In the illustrated embodiment, the longitudinal position of expandable member 454 has been maintained as in FIG. 13 while the profile of the expandable member 454 has been increased by inflating the expandable member 454 and causing the expandable member 454 to expand radially outward. The expandable member 454 can be enlarged to a greater extent in a central zone, as illustrated in FIG. 8A to induce a non-uniform diameter or width along the length of the scaffold 100. This condition is shown in the anatomy in FIG. 15.

After the scaffold 100 has been expanded and lodged into the urethra 14 the expandable member 454 can be un-expanded. The delivery catheter 408 can be retracted from the urethra 14 leaving the anchor catheter 404 in place. The position and expansion of the scaffold 100 can be verified. The elongate body 412 of the anchor catheter 404 could be used to deliver another catheter for a therapy within the prostate 10. For example, the expandable member 484 could be placed in the scaffold 100 and expanded to deliver a therapeutic agent to the prostate 10. The anchor catheter 404 could be used to bail out of the procedure. For example, the catheter 490 could be advanced over the elongate body 412 until the scoring features 496 are disposed in the scaffold 100. The expandable member 494 can be expanded to score inner surfaces of the scaffold to accelerate the breakdown of the scaffold. The anchor catheter 404 also can serve as a drainage catheter during recovery, allowing urine to pass through the lumen 434.

FIG. 15 shows that after any subsequent therapy is complete and after any recovery during which the anchor catheter 404 may be of assistance, the anchor catheter 404 is removed leaving the scaffold 100 in place. The scaffold 100 provides a therapy for an appropriate duration. As discussed above, the scaffold 100 can be a short term implant that is self-removing by dissolving in or reacting with urine. This process results in slow erosion that has minimal to no impact on the structural integrity of the scaffold until after the therapy is complete. Thereafter, the scaffold 100 can be fully expelled from the urethra 14 atraumatically. In other embodiments, the scaffolds disclosed herein do not dissolve, erode or otherwise begin to self-explant but remains in place indefinitely. In other embodiments, the scaffolds disclosed herein do not dissolve or erode or otherwise begin to self-explant until triggered, e.g., by the catheter 490 or by exposing the scaffold to a mild acid or other accelerant.

FIG. 15 shows that the scaffold 100 can be configured to not impeded flow in the seminal vesicle sv. The seminal vesicle is a conduit for semen and thus is important to sexual function. Blocking or jailing the seminal vesicle could be problematic particularly in longer term or permanent treatments. Accordingly, the wall pattern 150 can be modified to provide an enlarged gap at a location of the seminal vesicle sv. For instance, where undulating member 158 are provided in the pattern 150, one or more such members can be omitted creating a larger gap in the central zone 135. To achieve this, the connectors 162 can be elongated in that zone.

Placement of the scaffold 100 so modified can be by any suitable technique that provides some assurance of proper location and orientation of the scaffold 100 within the prostate. For example, where two slideable catheter bodies are provided as in FIG. 4, the distance from the balloon 428 to the expandable member 454 can be controlled to provide confirmation that the larger opening in the central zone 135 is aligned with the seminal vesicle sv. For example, the distal projection 470 can be sized based on the patient to locate this opening at the seminal vesicle sv. Further, the rotation al position of the connectors 162 can be confirmed to not be on the seminal vesicle sv by rotationally orienting a portion of the proximal end 446 relative to the connection hub 432 or another feature on the proximal end of the anchor catheter 404. A system with marker bands can also be used to position the scaffold 100 in the urethra 14 at the proper longitudinal and rotational position. The markers 520, 524 can be disposed on or adjacent to balloon 454 in the system 400C. The position of one or both of the markers 520, 524 can be aligned with the proximal and/or distal end of the prostate. When so aligned, an enlarged opening the wall pattern 150 disposed in the central zone 135 can be longitudinally aligned with the seminal vesicle sv. The location of the connectors 162 can be disposed on the delivery catheter 408C such that they are at least about 30 degrees from the center of the enlarged opening, at least about 60 degrees from the center of the enlarged opening, at least about 90 degrees from the center of the enlarged opening, or at least about 120 degrees from the center of the enlarged opening. In one embodiment, a single connector is provided between a distal portion of the central zone 135 and a proximal portion of the central zone 135 such that the opening extends from one side of the single connector 162, entirely around the circumference of the central zone 135 to the other side of the single connector 162. This provides for placement of the enlarged opening version of the scaffold 100 to permit the connector 162 to be 180 degrees from the seminal vesicle sv with no struts disposed circumferentially between this remote position and the opening of the seminal vesicle sv. While providing a larger opening is one approach, one could also use the delivery systems to carefully place the scaffold 100 with the wall pattern 150. One advantage of the scaffolds disclosed herein is the scaffolds can be easily and quickly removed by a trigger if the placement with respect to the seminal vesicle sv or otherwise is not appropriate.

An extended use embodiment of the scaffold is illustrated in the inset image of FIG. 3F. As discussed above, the outer portion 184 of the scaffold 100D covers and/or encloses the layer of magnesium or other urine soluble metal. The outer portion 184 is inert in urine such that the scaffold 100D remains intact for the duration of a therapy and can remain intact indefinitely. In one embodiment, the outer portion 184 is soluble in urine. The outer portion 184 can be soluble in urine at a slower rate than the inner portion 180. In one technique, the outer portion 184 is inert in urine but can be ruptured subsequent to implantation of the scaffold 100. For example after implantation the balloon catheter 490 can be delivered into the urethra 14 and into the space within the scaffold 100. The expandable member 494 can be expanded into engagement with the inside surface of the scaffold 100 then inflated to a pressure sufficient to cause the scoring feature 496 to rupture the outer portion 184. By rupturing the outer portion 184, access is provided for urine to flow through the outer portion 184 into the direct contact with the inner portion 180. This directed contact causes the inner portion 180 to begin to dissolve in the urine and to be carried out of the urethra 14. As such, removal of the scaffold 100D by absorption, dissolution, and/or erosion can be triggered. The outer portion 184 can be configured as a coating entirely encapsulating an inner portion comprising a magnesium alloy. The coating can be configure to prevent exposure of the magnesium alloy for a minimum of seven days, in some embodiments for a minimum of fourteen days, in some embodiments for at least twenty-one days, in other embodiments for at least third days, for other embodiments for at least sixty days and in other embodiments for at least ninety days when immersed in urine.

Although many embodiments disclosed herein can be removed by interactions between the scaffold and the body, e.g., by one or more of absorption into tissue surrounding the urethra 14, reaction with the urine, and by fracture and passing with the urine of segments of the scaffold. These processes can be expedited by a subsequent intervention. As discussed above in connection FIG. 9A, a balloon 480 can be placed in the urethra 14 to deliver a mild acid or other substance to more quickly reduce the thickness of the scaffold 100. The mild acid can flood the urethra 14 to act generally on the scaffold 100. In other techniques, the mild acid can elute in a prescribed pattern to focus the mild acid on specific locations of the scaffold. The mild acid can be eluted only at the proximal zone 131. The mild acid can be eluted only at the distal zone 129. The mild acid can be eluted only at the proximal zone 133 and the distal zone 129. The mild acid can be eluted only at the central zone 135.

FIG. 10 shows that removal of the scaffold 100E can be immediate rather than over time through a reaction or erosion by urine. An explantation system 550 can be inserted into the urethra 14. The system can include a sheath 554 and a snare 558. The snare 558 can include a proximal wire portion 562 that extends through a lumen of the sheath 554 and an arcuate portion 566. Relative motion can be provided between the snare 558 and the sheath 554. The snare 558 can be extended out of the sheath 554 such that the arcuate portion 566 is exposed. Thereafter the arcuate portion 566 can be advanced over or through the snare feature 199. The arcuate portion 566 can then be tightened over the snare feature 199 or otherwise engaged with the snare feature 199. Such engagement permits the distal face of the sheath 554 to be engaged with the proximal face of the scaffold 100E. As discussed above, the proximal angled faces of the scaffold 100E can abut the distal face of the sheath 554. When so engaged, further distal advancement of or pressure by the sheath 554 on the scaffold 100E causes the scaffold 100E to collapse and be drawn into the sheath 554 to be removed from the patient.

Although these inventions have been disclosed in the context of certain preferred embodiments and examples, it will be understood by those skilled in the art that the present inventions extend beyond the specifically disclosed embodiments to other alternative embodiments and/or uses of the inventions and obvious modifications and equivalents thereof. In addition, while several variations of the inventions have been shown and described in detail, other modifications, which are within the scope of these inventions, will be readily apparent to those of skill in the art based upon this disclosure. It is also contemplated that various combination or sub-combinations of the specific features and aspects of the embodiments may be made and still fall within the scope of the inventions. It should be understood that various features and aspects of the disclosed embodiments can be combined with or substituted for one another in order to form varying modes of the disclosed inventions. Thus, it is intended that the scope of at least some of the present inventions herein disclosed should not be limited by the particular disclosed embodiments described above.

### Example Embodiments

The following example embodiments identify some possible permutations of combinations of features disclosed herein, although other permutations of combinations of features are also possible.

### Example Embodiments

1. A scaffold for expanding a portion of a urethra that extends through a prostate, the scaffold comprising:
an elongate body having a proximal end, a distal end, an outer surface, and an inner surface,
the elongate body comprising a plurality of undulating circumferential members disposed between the proximal end and the distal end, the undulating circumferential members being spaced apart along a longitudinal axis of the scaffold and being connected to at least one adjacent undulating circumferential member by at least one axial connector;
the elongate body having a collapsed state and an expanded state, the collapsed state configured to enable the elongate body to be delivered into the urethra and to enable the elongate body to be navigated to a position within the portion of the urethra that extends through the prostate, the expanded state configured such that the undulating circumferential members provide the elongate body with sufficient radial strength to maintain open the portion of the urethra that extends through the prostate,
the elongate body having a width defined in a plane transverse to the longitudinal axis at a distal-end, the elongate body having a width defined in a plane transverse to the longitudinal axis at a central section located between the distal end and the proximal end, the width at the central section exceeding the width at the distal end, and
wherein the elongate body comprises a material that reacts with urine or with the tissue surrounding the urethra to reduce the volume of the elongate body such that the scaffold can be removed from the urethra after a prescribed period without requiring an interventional procedure.

2. The scaffold of Embodiment 1, wherein at least one of the undulating members comprise a proximal apex, a distal apex, and an elongate member extending between the proximal apex and the distal apex, the elongate member comprising a necked down region.

3. The scaffold of Embodiment 2, wherein the necked down region comprises a strut width that is less than a strut width of at least one of the proximal apex and the distal apex.

4. The scaffold of Embodiment 2, wherein the necked down region comprises a strut thickness that is less than a strut thickness of at least one of the proximal apex and the distal apex.

5. The scaffold of Embodiment 2, wherein the necked down region comprises at least one aperture formed through the elongate body from the inner surface to the outer surface.

6. The scaffold of Embodiment 5, wherein the necked down region comprises a plurality of apertures formed therethrough.

7. The scaffold of Embodiment 1, wherein the material comprises magnesium.

8. The scaffold of Embodiment 1, wherein the material comprises magnesium configured to react with urine to cause the elongate body to fracture within 30 days when exposed to urine.

9. A scaffold for expanding the urethra, comprising:
an elongate body having a proximal end, a distal end, an outer surface, and an inner surface,
the elongate body being disposed along a longitudinal axis of the scaffold between the proximal end and the distal end, the elongate body having a collapsed state and an expanded state, the elongate body having sufficient radial strength to maintain a lumen thereof open when disposed in the urethra,
the outer surface of elongate body having a width defined in a plane transverse to the longitudinal axis at a distal-end, the outer surface of elongate body having a width defined in a plane transverse to the longitudinal axis at a central section located between the distal end and the proximal end, the width at the central section exceeding the width at the distal end,
wherein the elongate body comprises a removal configuration in which the scaffold will self-explant after a prescribed period.

10. The scaffold of Embodiment 9, further comprising a material enabling the scaffold to be removed from the urethra after a prescribed period by interaction with urine in the urethra, with tissues disposed around the urethra or after a shortened period upon exposure to an external agent.

11. The scaffold of Embodiment 10, wherein the material comprises magnesium.

12. The scaffold of Embodiment 9, wherein the elongate body comprises an inner layer comparing a first material that is reactive with urine or the tissue surrounding the urethra and an outer layer comprising a second material that is less reactive with urine or with the tissue surrounding the urethra than the first material.

13. The scaffold of Embodiment 12, wherein the first material comprises magnesium and the second material comprises an absorbable polymer.

14. The scaffold of Embodiment 9, wherein the outer layer completely encapsulates the inner layer.

15. The scaffold of Embodiment 9, wherein the elongate body has at least one necked down region configured to cause the scaffold to fracture into at least two segments, the segments being sized to pass out of the urethra with urine flow.

16. The scaffold of Embodiment 15, wherein the necked down region is disposed between the central section and the distal end.

17. The scaffold of Embodiment 15, wherein a first necked down region is disposed between the central section and the distal end and a second necked down region is disposed between the central section and the proximal end.

18. A scaffold for expanding a urethra through a narrows formed in the urethra, comprising:
an elongate body having a proximal end, a distal end, an outer surface to be expanded into contact with the urethra, and an inner surface configured to surround a lumen for urine flow,
the elongate body having an expanded state configured with sufficient radial strength to maintain the lumen open when disposed in the narrows of the urethra, wherein at least a portion of the elongate body is configured to be eroded by urine.

19. The scaffold of Embodiment 18, wherein the elongate body has a first width defined in a plane transverse to the longitudinal axis at a distal end and a second width defined in a plane transverse to the longitudinal axis at a central section located between the distal end and the proximal end, the second width being greater than the first width.

20. The scaffold of Embodiment 18, wherein the elongate body comprises a material including magnesium, the material configured to be eroded by urine.

21. The scaffold of Embodiment 20, wherein the material including magnesium is disposed in an inner portion surrounded by an outer portion, the outer portion being eroded by urine at a lower rate the material configured to be eroded by urine.

22. The scaffold of Embodiment 21, wherein the outer portion is configured to be modified by a subsequent intervention to expose inner portion to urine.

23. A method comprising:
advancing into a urethra of a patient a delivery catheter comprising a scaffold coupled therewith, the scaffold having a proximal end, a distal end, and an elongate body disposed therebetween, the elongate body including a central portion of the scaffold;
positioning the scaffold such that the distal end is adjacent to an end of the prostate closest to the bladder and the proximal end is adjacent to an end of the prostate farthest away from the bladder;
confirming the position of the scaffold within the prostate; and
expanding the scaffold away from a longitudinal axis of the catheter into apposition with the tissue surrounding the urethra; and
enlarging the central portion of the elongate body by a larger amount than at least one of the proximal end and the distal end of the scaffold;
whereby the constriction of the urethra within the prostate is reduced.

24. The method of Embodiment 21, wherein confirming the position comprises advancing a balloon into the bladder; expanding the balloon and retracting the balloon into engagement with a neck of the bladder; moving a distal portion of the delivery catheter into contact with a proximal face of the balloon.

25. The method of Embodiment 21, wherein confirming the position comprises viewing the position of one or more marker bands disposed at a known position relative to the scaffold.

26. The method of Embodiment 21, wherein expanding comprises expanding a plurality of undulating circumferential members spaced apart along the length of the scaffold.

27. A system for treating obstruction of a urethra, comprising:
a scaffold delivery assembly comprising a scaffold delivery catheter comprising an elongate body having a proximal end, a distal end, a central lumen disposed between the proximal and distal ends, and a deployment balloon disposed on a side surface of the elongate body, the deployment balloon being in fluid communication with an inflation lumen disposed in the elongate scaffold delivery catheter body between the proximal end and the deployment balloon; and
a temporary urethral scaffold comprising a material configured to react with urine and/or tissue disposed around the urethra to cause the scaffold to erode over time and to self-explant after a prescribed period.

28. The scaffold deployment system of Embodiment 25, further comprising an anchor balloon disposed at a distal end of the scaffold deployment system.

29. The scaffold deployment system of Embodiment 26, wherein the anchor balloon is disposed at a distal end of an anchor balloon catheter, the anchor balloon catheter having an elongate body coupled with the anchor balloon and extending proximally therefrom, the elongate body slideably disposed in a lumen of the scaffold delivery catheter and configured to convey inflation media to the anchor balloon.

30. The scaffold deployment system of Embodiment 27, wherein the anchor balloon catheter comprises a lumen extending from a distal end to a proximal end thereof to convey urine out of the patient during a procedure or during recovery.

31. A method for explanting a urethral scaffold, comprising:
advancing a distal portion of an elongate catheter body into a urethra;
advancing the distal portion of the elongate catheter body into a lumen of a scaffold disposed within a portion of the urethra, the scaffold having an outer surface disposed against the urethra and an inner surface defining a lumen for urine flow; and
activating the distal portion of the elongate catheter body to initiate or to accelerate erosion of the scaffold.

32. The method of Embodiment 29, wherein the distal portion of the elongate catheter body includes an expandable member and at least one scoring feature and activating includes expanding the expandable member to score the inner surface of the scaffold.

33. The method of Embodiment 30, wherein the scoring features are disposed in a proximal portion of the elongate member.

34. The method of Embodiment 31, wherein the scoring features are disposed in a distal portion of the elongate member.

35. The method of Embodiment 29, wherein activating includes delivering an erosion accelerant through the distal portion of the elongate catheter body into the urethra adjacent to the inner surface of the scaffold.

36. The method of Embodiment 33, wherein delivering the erosion accelerant include inflating a balloon with an inflation medium disposed on the distal portion of the elongate catheter body and maintaining pressure in the balloon while the inflation medium flows out of a surface of the balloon onto the inner surface of the scaffold.

37. A method for explanting a urethral scaffold, comprising:
advancing a distal portion of an elongate catheter body into a urethra, the elongate catheter body having a distal portion and a lumen therein, a snare extending within the lumen;
advancing the distal portion of the elongate catheter body adjacent to a proximal end of a scaffold disposed within a portion of the urethra, the scaffold having an outer surface disposed against the urethra and a snare feature disposed inwards of the outer surface; and
advancing an arcuate portion of the snare from the elongate catheter body;
engaging the arcuate feature with the snare of the scaffold; and
providing relative motion between the snare of the scaffold and the distal portion of the elongate catheter body to compress the scaffold into the lumen of the elongate catheter body.

38. The method of Embodiment 35, wherein the scaffold comprises a tapered proximal portion configured to be at least partially received in the lumen of the elongate catheter body before the scaffold begins to compress.

39. The scaffold of Embodiment 7, wherein the material is selected from the group consisting of magnesium, magnesium alloy, iron, zinc, PLLA, PLGA, and compounds that can be plastically deformed to trigger a degradation of the compound in vivo.

40. The system of Embodiment 25, further comprising a first marker longitudinally disposed between the proximal end of the scaffold delivery catheter and the deployment balloon, the first marker comprising a radiopaque material.

41. The system of Embodiment 38, further comprising a second marker longitudinally disposed between the distal end of the scaffold delivery catheter and the deployment balloon, the second marker comprising a radiopaque material.

What is claimed is:

1. A scaffold for expanding a portion of a urethra that extends through a prostate, the scaffold comprising:
an elongate body having a proximal end, a distal end, an outer surface, and an inner surface,
the elongate body comprising a plurality of undulating circumferential members disposed between the proximal end and the distal end, the undulating circumferential members being spaced apart along a longitudinal axis of the scaffold and being connected to at least one adjacent undulating circumferential member by at least one axial connector;
the elongate body having a collapsed state and an expanded state, the collapsed state configured to enable the elongate body to be delivered into the urethra and to enable the elongate body to be navigated to a position within the portion of the urethra that extends through the prostate, the expanded state configured such that the undulating circumferential members provide the elongate body with sufficient radial strength to maintain open the portion of the urethra that extends through the prostate,
the elongate body having a width defined in a plane transverse to the longitudinal axis at a distal-end, the elongate body having a width defined in a plane transverse to the longitudinal axis at a central section located between the distal end and the proximal end, the width at the central section exceeding the width at the distal end, and
wherein the elongate body comprises a material that reacts with urine or with the tissue surrounding the urethra to reduce the volume of the elongate body such that the scaffold can be removed from the urethra after a prescribed period without requiring an interventional procedure.

2. The scaffold of claim 1, wherein at least one of the undulating members comprise a proximal apex, a distal apex, and an elongate member extending between the proximal apex and the distal apex, the elongate member comprising a necked down region.

3. The scaffold of claim 2, wherein the necked down region comprises a strut width that is less than a strut width of at least one of the proximal apex and the distal apex.

4. The scaffold of claim 2, wherein the necked down region comprises a strut thickness that is less than a strut thickness of at least one of the proximal apex and the distal apex.

5. The scaffold of claim 2, wherein the necked down region comprises at least one aperture formed through the elongate body from the inner surface to the outer surface.

6. The scaffold of claim 5, wherein the necked down region comprises a plurality of apertures formed therethrough.

7. The scaffold of claim 1, wherein the material comprises magnesium.

8. The scaffold of claim 1, wherein the material comprises magnesium configured to react with urine to cause the elongate body to fracture within 30 days when exposed to urine.

9. A scaffold for expanding the urethra, comprising:
an elongate body having a proximal end, a distal end, an outer surface, and an inner surface,
the elongate body being disposed along a longitudinal axis of the scaffold between the proximal end and the distal end, the elongate body having a collapsed state and an expanded state, the elongate body having sufficient radial strength to maintain a lumen thereof open when disposed in the urethra,
the outer surface of elongate body having a width defined in a plane transverse to the longitudinal axis at a distal-end, the outer surface of elongate body having a width defined in a plane transverse to the longitudinal axis at a central section located between the distal end and the proximal end, the width at the central section exceeding the width at the distal end,
wherein the elongate body comprises a removal configuration in which the scaffold will self-explant after a prescribed period.

10. The scaffold of claim 9, further comprising a material enabling the scaffold to be removed from the urethra after a prescribed period by interaction with urine in the urethra, with tissues disposed around the urethra or after a shortened period upon exposure to an external agent.

11. The scaffold of claim 10, wherein the material comprises magnesium.

12. The scaffold of claim 9, wherein the elongate body comprises an inner layer comparing a first material that is reactive with urine or the tissue surrounding the urethra and an outer layer comprising a second material that is less reactive with urine or with the tissue surrounding the urethra than the first material.

13. The scaffold of claim 12, wherein the first material comprises magnesium and the second material comprises an absorbable polymer.

14. The scaffold of claim 9, wherein the outer layer completely encapsulates the inner layer.

15. The scaffold of claim 9, wherein the elongate body has at least one necked down region configured to cause the scaffold to fracture into at least two segments, the segments being sized to pass out of the urethra with urine flow.

16. The scaffold of claim 15, wherein the necked down region is disposed between the central section and the distal end.

17. The scaffold of claim 15, wherein a first necked down region is disposed between the central section and the distal end and a second necked down region is disposed between the central section and the proximal end.

18. A scaffold for expanding a urethra through a narrows formed in the urethra, comprising:
an elongate body having a proximal end, a distal end, an outer surface to be expanded into contact with the urethra, and an inner surface configured to surround a lumen for urine flow,
the elongate body having an expanded state configured with sufficient radial strength to maintain the lumen open when disposed in the narrows of the urethra, wherein at least a portion of the elongate body is configured to be eroded by urine.

19. The scaffold of claim 18, wherein the elongate body has a first width defined in a plane transverse to the longitudinal axis at a distal end and a second width defined in a plane transverse to the longitudinal axis at a central section located between the distal end and the proximal end, the second width being greater than the first width.

20. The scaffold of claim 18, wherein the elongate body comprises a material including magnesium, the material configured to be eroded by urine.

21. The scaffold of claim 20, wherein the material including magnesium is disposed in an inner portion surrounded by an outer portion, the outer portion being eroded by urine at a lower rate than the material configured to be eroded by urine.

22. The scaffold of claim 21, wherein the outer portion is configured to be modified by a subsequent intervention to expose the inner portion to urine.

23. The scaffold of claim 1, wherein the material is selected from the group consisting of iron, zinc, PLLA, PLGA, and compounds that can be plastically deformed to trigger a degradation of the compound in vivo.
